(19) United States
(12) Patent Application Publication
(10) Pub. No.: US 2025/0339610 A1
(43) Pub. Date: Nov. 6, 2025

(54) SUBCUTANEOUS MEDICAL DEVICE SYSTEM

(71) Applicant: Unomedical A/S, Lejre (DK)

(72) Inventors: Chih-Ho Chen, Taipei City (TW); Hung-Hsiang Chen, Boston, MA (US)

(21) Appl. No.: 19/266,240

(22) Filed: Jul. 11, 2025

Related U.S. Application Data
(63) Continuation of application No. PCT/EP2024/050553, filed on Jan. 11, 2024.
(60) Provisional application No. 63/438,818, filed on Jan. 13, 2023.
(60) Provisional application No. 63/438,846, filed on Jan. 13, 2023.
(60) Provisional application No. 63/438,855, filed on Jan. 13, 2023.

(30) Foreign Application Priority Data
Mar. 1, 2023 (GB) 2302983.8
Mar. 1, 2023 (GB) 2302984.6
Mar. 1, 2023 (GB) 2302985.3

(51) Int. Cl. A61M 5/142

(52) U.S. Cl. CPC A61M 5/14248; A61M 2205/3303

(57) ABSTRACT

The present invention provides a subcutaneous medical device system, comprising an interface patch and a subcutaneous medical device having an adhesive surface and a needle for piercing a patient's skin. The interface patch comprises: a proximal side having an adhesive region for attaching the interface patch to the patient's skin; a distal side to which the adhesive surface of the subcutaneous medical device is attachable. The interface patch also includes an aperture or pierceable region positioned to receive the needle of the subcutaneous medical device such that when the subcutaneous medical device is attached to the interface patch the needle can pierce the patient's skin through the aperture or pierceable region.

The invention relates generally to a subcutaneous medical device system.

### BACKGROUND

Subcutaneous medical devices, such as glucose sensors and infusion sets, are worn by patients for prolonged periods of time (e.g., days or weeks) and include a needle that is subcutaneously positioned, e.g., to detect blood glucose or deliver insulin. These devices typically have a small and discreet housing that is attached to a patient by an adhesive patch. The adhesive patch is attached to the housing and is located between the housing and the skin when applied, and acts to seal the puncture site of the needle.

As these devices are worn for prolonged periods of time, the adhesive patch typically has a strong, water-resistant adhesive to prevent the device from moving or coming detached and to maintain protection of the skin puncture site.

However, removal of these devices, particularly the adhesive patch, can cause skin irritation and damage, such as medical adhesive related skin injuries. This can include tension injuries caused by pulling the skin during removal, blisters, skin tears, skin stripping, irritant contact dermatitis, maceration, and folliculitis.

Additionally, some patients wear an additional covering dressing that covers the device (housing and adhesive patch) to protect it from knocks, water and dirt.

These additional dressings are applied separately to the device and are also adhered to the skin and also cause skin irritation and damage.

It would be desirable to provide an apparatus that addresses at least some of the above problems.

### SUMMARY OF THE INVENTION

In accordance with the present invention there is provided a subcutaneous medical device system according to the appended claims.

According to the invention, there is provided a subcutaneous medical device system, comprising an interface patch and a subcutaneous medical device having an adhesive surface and a needle for piercing a patient's skin, wherein the interface patch comprises: a proximal side having an adhesive region for attaching the interface patch to the patient's skin; a distal side to which the adhesive surface of the subcutaneous medical device is attachable; and an aperture or pierceable region positioned to receive the needle of the subcutaneous medical device such that when the subcutaneous medical device is attached to the interface patch the needle can pierce the patient's skin through the aperture or pierceable region.

Thus, the interface patch is provided between the patient's skin and the adhesive surface of the subcutaneous medical device so that the subcutaneous medical device adheres to the interface patch rather than directly onto the skin. By providing an interface patch in this way, skin irritation and damage is reduced on removal of the subcutaneous medical device system from the patient.

In examples, the pierceable region may be thinner compared to other areas of the interface patch, or may be formed from a different material.

In examples, the interface patch is larger than the adhesive surface of the subcutaneous medical device. In examples, the adhesive region on the proximal side of the interface patch has a larger surface area than the adhesive surface of the subcutaneous medical device.

In examples, the adhesion strength of the adhesive region on the proximal side is less than the adhesion strength of the adhesive surface of the subcutaneous medical device. That is, the adhesive on the proximal side of the interface patch is weaker per unit area than the adhesive on the adhesive surface of the subcutaneous medical device. Therefore, the interface patch can use a less strong adhesive than that on subcutaneous medical device to provide the same overall attachment force, and make removal of the interface patch from the skin less damaging to the skin than removal of the subcutaneous medical device directly from the skin.

In examples, the adhesive region on the proximal side of the interface patch comprises a uniform adhesion surface. In examples, the adhesive region on the proximal side of the interface patch is pattern-applied. In some examples, the adhesive region comprises a line pattern. In other examples, the adhesive region comprises a grid pattern. Providing a pattern-applied adhesive can further reduce damage to the skin on removal of the interface patch from the skin.

In examples, the adhesive region on the proximal side of the interface patch comprises at least one region of reduced adhesion. The at least one region of reduced adhesion may be disposed at a pull tab region of the interface patch and may reduce skin irritation and damage on removal of the interface patch from the patient's skin.

In examples, the interface patch further comprises at least one removable release layer covering the adhesive region. The release layer is removed before applying the interface patch to the patient's skin. In examples, the release layer is a single piece release layer. In examples, the single piece release layer comprises a first end at the edge of the proximal side and a second end at the aperture. In examples, the single piece release layer has a spiral form between the first end and the second end.

In examples, the release layer comprises a plurality of sections. In examples, the release layer comprises a central section covering a central region of the proximal side of the interface patch, and a peripheral section covering an edge region of the proximal side of the interface patch.

In examples, the at least one release layer comprises a pull tab or pull tab region. In examples, the interface patch comprises a polygonal shape and the pull tab or pull tab region may be located at a corner of the interface patch.

In examples, the interface patch further comprises at least one support layer attached to the distal side of the interface patch. In examples, the support layer is removable. The support layer may increase the stiffness of the interface patch during application, for example to reduce wrinkling and folding of the interface patch.

In examples, the subcutaneous medical device is a glucose monitor. In some examples, the glucose monitor is a flash (i.e. intermittent) glucose monitor. In other examples, the glucose monitor is a continuous glucose monitor.

In examples, the subcutaneous medical device is an infusion set. The infusion set may comprise a connector for connecting the infusion set to a source of medicament, e.g., a medicament pump. The infusion set may be for providing insulin to the patient.

In examples, the needle extends in a direction perpendicular to the adhesive surface of the subcutaneous medical device.

In examples, the subcutaneous medical device further comprises a cover attachable to the interface patch to cover the subcutaneous medical device. In examples, the cover is concave. By providing a cover, the subcutaneous medical device can be protected from contact, knocking and contamination.

According to the invention, there is also provided a method of using the subcutaneous medical device system described above. The method may comprise: applying the interface patch to the patient's skin, and applying the subcutaneous medical device such that the needle pierces the patient's skin through the aperture of the interface patch. According to the invention, there is provided an interface patch for a subcutaneous medical device having an adhesive surface and a needle for piercing a patient's skin, wherein the interface patch comprises: a proximal side having an adhesive region for attaching the interface patch to the patient's skin; a distal side to which the adhesive surface of the subcutaneous medical device is attachable; and an aperture positioned to receive the needle of the subcutaneous medical device such that when the subcutaneous medical device is attached to the interface patch the needle can pierce the patient's skin through the aperture or pierceable region.

Thus, the interface patch is provided between the patient's skin and the adhesive surface of the subcutaneous medical device so that the subcutaneous medical device adheres to the interface patch rather than directly onto the skin. By providing an interface patch in this way, skin irritation and damage is reduced on removal of the subcutaneous medical device system from the patient.

In examples, the pierceable region may be thinner compared to other areas of the interface patch, or may be formed from a different material.

In examples, the interface patch is larger than the adhesive surface of the subcutaneous medical device. In examples, the adhesive region on the proximal side of the interface patch has a larger surface area than the adhesive surface of the subcutaneous medical device.

In examples, the adhesion strength of the adhesive region on the proximal side is less than the adhesion strength of the adhesive surface of the subcutaneous medical device. That is, the adhesive on the proximal side of the interface patch is weaker per unit area than the adhesive on the adhesive surface of the subcutaneous medical device. Therefore, the interface patch can use a less strong adhesive than that on subcutaneous medical device to provide the same overall attachment force, and make removal of the interface patch from the skin less damaging to the skin than removal of the subcutaneous medical device directly from the skin.

In examples, the adhesive region on the proximal side of the interface patch comprises a uniform adhesion surface. In examples, the adhesive region on the proximal side of the interface patch is pattern-applied. In some examples, the adhesive region comprises a line pattern. In other examples, the adhesive region comprises a grid pattern. Providing a pattern-applied adhesive can further reduce damage to the skin on removal of the interface patch from the skin.

In examples, the adhesive region on the proximal side of the interface patch comprises at least one region of reduced adhesion. The at least one region of reduced adhesion may be disposed at a pull tab region of the interface patch and may reduce skin irritation and damage on removal of the interface patch from the patient's skin.

In examples, the interface patch further comprises at least one removable release layer covering the adhesive region. The release layer is removed before applying the interface patch to the patient's skin. In examples, the release layer is a single piece release layer. In examples, the single piece release layer comprises a first end at the edge of the proximal side and a second end at the aperture. In examples, the single piece release layer has a spiral form between the first end and the second end.

In examples, the release layer comprises a plurality of sections. In examples, the release layer comprises a central section covering a central region of the proximal side of the interface patch, and a peripheral section covering an edge region of the proximal side of the interface patch.

In examples, the at least one release layer comprises a pull tab or pull tab region. In examples, the interface patch comprises a polygonal shape and the pull tab or pull tab region may be located at a corner of the interface patch.

In examples, the interface patch further comprises at least one support layer attached to the distal side of the interface patch. In examples, the support layer is removable. The support layer may increase the stiffness of the interface patch during application, for example to reduce wrinkling and folding of the interface patch.

According to the invention, there is also provided a subcutaneous medical device system for attaching to a patient's skin, comprising: a subcutaneous medical device comprising an adhesive surface and a needle for piercing a patient's skin; and an interface patch. The interface patch may be as described above.

In examples, the subcutaneous medical device is a glucose monitor. In some examples, the glucose monitor is a flash (i.e. intermittent) glucose monitor. In other examples, the glucose monitor is a continuous glucose monitor.

In examples, the subcutaneous medical device is an infusion set. The infusion set may comprise a connector for connecting the infusion set to a source of medicament, e.g., a medicament pump. The infusion set may be for providing insulin to the patient.

In examples, the needle extends in a direction perpendicular to the adhesive surface of the subcutaneous medical device.

In examples, the subcutaneous medical device further comprises a cover attachable to the interface patch to cover the subcutaneous medical device. In examples, the cover is concave. By providing a cover, the subcutaneous medical device can be protected from contact, knocking and contamination.

According to the invention, there is also provided a method of using the interface patch described above for attaching a subcutaneous medical device to a patient's skin. The method may comprise: applying the interface patch to the patient's skin, and applying the subcutaneous medical device such that the needle pierces the patient's skin through the aperture of the interface patch.

According to the invention, there is provided a subcutaneous medical device system, comprising an interface patch and a subcutaneous medical device having an adhesive surface and a needle for piercing a patient's skin, wherein the interface patch comprises: a first portion having a proximal side with an adhesive for attaching the interface patch to the patient's skin, and a distal side to which the adhesive surface of the subcutaneous medical device can be attached such that when the subcutaneous medical device is attached to the interface patch the needle can pierce the patient's skin; and a second portion, extending from the first portion and foldable to cover the subcutaneous medical device when the subcutaneous medical device is adhered to the first portion.

Thus, the second portion of the interface patch is folded over the subcutaneous medical device and acts to improve protection the subcutaneous medical device (e.g., against knocks, water, and dirt), and may make the subcutaneous medical device more discreet.

In examples, the first portion is joined to the second portion at a neck. In examples, the neck is narrower than the first portion and the second portion.

In examples, the first portion has a generally triangular shape.

In examples, the second portion has a generally triangular shape.

In examples, the first portion has a generally triangular shape and the second portion has a generally triangular shape. In examples, the first portion and the second portion are joined at corners of the first and second portions such that the first and second portions have opposite orientations before the second portion is folded to cover the subcutaneous medical device.

In examples, the second portion is smaller than the first portion. Accordingly, the second portion only adheres to the first portion and does not adhere to the patient's skin.

In examples, a distal side of the second portion comprises an adhesive for adhering to the distal side of the first portion when the second portion is folded to cover the subcutaneous medical device.

In examples, the second portion is flexible.

In examples, the interface patch further comprises a rigid cover arranged to cover the subcutaneous medical device when the second portion is folded to cover the subcutaneous medical device. In examples, the rigid cover is attached to the second portion. In examples, the rigid cover is concave.

In examples, the first portion comprises a pull tab region for removal of the first portion from the patient's skin. In examples, the first portion comprises a region of reduced adhesion disposed adjacent to the pull tab region. In examples, the region of reduced adhesion comprises a first region disposed adjacent to the pull tab region, and at least one line extending from the first portion. In examples, the region of reduced adhesion comprises first and second lines extending away from the first region in a divergent manner. In examples, the region of reduced adhesion further comprises at least one arm extending from the at least one line. In examples, the region of reduced adhesion comprises no adhesive, or a reduced thickness of adhesive, or a different, less strong adhesive.

In examples, the subcutaneous medical device comprises a device body and an adhesive patch for adhering the device body to the interface patch.

In examples, the subcutaneous medical device further comprises an applicator for applying the subcutaneous medical device to the interface patch and patient.

In examples, the subcutaneous medical device comprises a glucose sensor, for example a flash glucose sensor or a continuous glucose monitor.

In examples, the subcutaneous medical device comprises an infusion set for delivery of a medicament to the patient.

According to the invention, there is also provided a method of using the subcutaneous medical device system, which comprises the steps of: attaching the proximal side of the first portion of the interface patch to the patient's skin; attaching the subcutaneous medical device to the distal side of the first portion of the interface patch; and folding the second portion to cover the subcutaneous medical device. In examples, the method may comprise attaching the subcutaneous medical device to the distal side of the first portion of the interface patch using an applicator.

According to the invention, there is provided an interface patch for a subcutaneous medical device having an adhesive surface and a needle for piercing a patient's skin, wherein the interface patch comprises: a first portion having a proximal side with an adhesive for attaching the interface patch to the patient's skin, and a distal side to which the adhesive surface of the subcutaneous medical device can be attached such that when the subcutaneous medical device is attached to the interface patch the needle can pierce the patient's skin; and a second portion, extending from the first portion and foldable to cover the subcutaneous medical device when the subcutaneous medical device is adhered to the first portion.

Then the second portion of the interface patch is folded over the subcutaneous medical device it acts to improve protection the subcutaneous medical device (e.g., against knocks, water, and dirt), and may make the subcutaneous medical device more discreet.

In examples, the first portion is joined to the second portion at a neck. In examples, the neck is narrower than the first portion and the second portion.

In examples, the first portion has a generally triangular shape.

In examples, the second portion has a generally triangular shape.

In examples, the first portion has a generally triangular shape and the second portion has a generally triangular shape. In examples, the first portion and the second portion are joined at corners of the first and second portions such that the first and second portions have opposite orientations before the second portion is folded to cover the subcutaneous medical device.

In examples, the second portion is smaller than the first portion. Accordingly, the second portion only adheres to the first portion and does not adhere to the patient's skin.

In examples, a distal side of the second portion comprises an adhesive for adhering to the distal side of the first portion when the second portion is folded to cover the subcutaneous medical device.

In examples, the second portion is flexible.

In examples, the interface patch further comprises a rigid cover arranged to cover the subcutaneous medical device when the second portion is folded to cover the subcutaneous medical device. In examples, the rigid cover is attached to the second portion. In examples, the rigid cover is concave.

In examples, the first portion comprises a pull tab region for removal of the first portion from the patient's skin. In examples, the first portion comprises a region of reduced adhesion disposed adjacent to the pull tab region. In examples, the region of reduced adhesion comprises a first region disposed adjacent to the pull tab region, and at least one line extending from the first portion. In examples, the region of reduced adhesion comprises first and second lines extending away from the first region in a divergent manner. In examples, the region of reduced adhesion further comprises at least one arm extending from the at least one line. In examples, the region of reduced adhesion comprises no adhesive, or a reduced thickness of adhesive, or a different, less strong adhesive.

According to the invention, there is also provided a subcutaneous medical device system for attaching to a patient's skin, comprising a subcutaneous medical device comprising an adhesive surface and a needle for piercing a patient's skin, and an interface patch as described above.

In examples, the subcutaneous medical device comprises a device body and an adhesive patch for adhering the device body to the interface patch.

In examples, the subcutaneous medical device further comprises an applicator for applying the subcutaneous medical device to the interface patch and patient.

In examples, the subcutaneous medical device comprises a glucose sensor, for example a flash glucose sensor or a continuous glucose monitor.

In examples, the subcutaneous medical device comprises an infusion set for delivery of a medicament to the patient.

According to the invention, there is also provided a method of using the interface patch for attaching the subcutaneous medical device to a patient's skin, which comprises the steps of: attaching the proximal side of the first portion of the interface patch to the patient's skin; attaching the subcutaneous medical device to the distal side of the first portion of the interface patch; and folding the second portion to cover the subcutaneous medical device. In examples, the method may comprise attaching the subcutaneous medical device to the distal side of the first portion of the interface patch using an applicator.

According to the invention, there is provided a subcutaneous medical device system, comprising an interface patch and a subcutaneous medical device having an adhesive surface and a needle for piercing a patient's skin, wherein the interface patch comprises: a proximal side with an adhesive region for attaching the interface patch to the patient's skin, a distal side to which the adhesive surface of the subcutaneous medical device can be attached such that when the subcutaneous medical device is attached to the interface patch the needle can pierce the patient's skin; and a pull tab region for removing the interface patch from the patient's skin; wherein the adhesive region comprises a region of reduced adhesion comprising a first region disposed adjacent to the pull tab region and at least one line extending from the first region.

The region of reduced adhesion is adhered to the patient's skin with reduced strength compared to the remainder of the adhesive region. The region of reduced adhesion, in particular the first region and the at least one line, act to reduce skin tension as the interface patch is peeled from the patient's skin on removal. This can make removal of the interface patch easier and reduce irritation and damage to the patient's skin

In examples, the region of reduced adhesion comprises a first line and a second line extending from the first region in a divergent manner.

In examples, the region of reduced adhesion is adhesive-free. In other examples, the region of reduced adhesion comprises a lower adhesive thickness than the remainder of the adhesive region.

In examples, the adhesive region comprises a pattern-applied adhesive. In some examples, the pattern-applied adhesive is a pattern-applied pressure sensitive adhesive.

In examples, the adhesive region extends to a peripheral edge of the interface patch.

In examples, the pull tab region is adhesive-free.

In examples, the pull tab region extends from an edge of the interface patch. The pull tab region may be defined on a distinct pull tab extending from a peripheral edge of the interface patch. The interface patch may be circular or oval, having a single edge.

In examples, the interface patch comprises a plurality of pull tab regions and a plurality of regions of reduced adhesion, each region of reduced adhesion being disposed adjacent to one of the plurality of pull tab regions.

In examples, the interface patch has a polygonal shape, with sides and corners. In examples, the interface patch has a triangular shape with three sides and three corners.

In examples, the pull tab region is formed at a corner of the interface patch. In examples, the interface patch comprises a plurality of regions of reduced adhesion, each region of reduced adhesion being disposed adjacent to a corner of the interface patch, and wherein a plurality of pull tab regions are formed at the respective corners. The interface patch may comprises one or more indications on the distal side to indicate the location of a pull tab region.

In examples, the interface patch further comprises an aperture for the needle of the subcutaneous medical device. In examples, the interface patch comprises a non-adhesive region surrounding the aperture.

In examples, the interface patch comprises: a first portion comprising the adhesive region, wherein the subcutaneous medical device can be adhered to the first patch portion, and a second portion extending from the first portion and foldable to cover the subcutaneous medical device when the subcutaneous medical device is adhered to the first portion. In examples, the first portion is joined to the second portion at a neck.

In examples, the first portion has a polygonal shape, for example a triangular shape.

In examples, the second portion is smaller than the first portion.

In examples, the second portion comprises an adhesive for adhering to the first portion when the second portion is folded over the first portion.

In examples, the subcutaneous medical device comprises a device body and an adhesive patch for adhering the device body to the interface patch.

In examples, the subcutaneous medical device comprises a glucose sensor, for example a flash glucose sensor or a continuous glucose monitor.

In examples, the subcutaneous medical device comprises an infusion set for delivery of a medicament to the patient.

According to the invention, there is also provided method of using the subcutaneous medical device system. The method comprises: applying the interface patch to the patient's skin, attaching the subcutaneous medical device to the interface patch; and removing the interface patch from the patient's skin using the pull tab region.

According to the invention, there is provided an interface patch for a subcutaneous medical device having an adhesive surface and a needle for piercing a patient's skin, wherein the interface patch comprises: a proximal side with an adhesive region for attaching the interface patch to the patient's skin, a distal side to which the adhesive surface of the subcutaneous medical device can be attached such that when the subcutaneous medical device is attached to the interface patch the needle can pierce the patient's skin; and a pull tab region for removing the interface patch from the patient's skin; wherein the adhesive region comprises a region of reduced adhesion comprising a first region disposed adjacent to the pull tab region and at least one line extending from the first region.

The region of reduced adhesion is adhered to the patient's skin with reduced strength compared to the remainder of the adhesive region. The region of reduced adhesion, in particular the first region and the at least one line, act to reduce skin tension as the interface patch is peeled from the patient's skin on removal. This can make removal of the interface patch easier and reduce irritation and damage to the patient's skin

In examples, the region of reduced adhesion comprises a first line and a second line extending from the first region in a divergent manner.

In examples, the region of reduced adhesion is adhesive-free. In other examples, the region of reduced adhesion comprises a lower adhesive thickness than the remainder of the adhesive region.

In examples, the adhesive region comprises a pattern-applied adhesive. In some examples, the pattern-applied adhesive is a pattern-applied pressure sensitive adhesive.

In examples, the adhesive region extends to a peripheral edge of the interface patch.

In examples, the pull tab region is adhesive-free.

In examples, the pull tab region extends from an edge of the interface patch. The pull tab region may be defined on a distinct pull tab extending from a peripheral edge of the interface patch. The interface patch may be circular or oval, having a single edge.

In examples, the interface patch comprises a plurality of pull tab regions and a plurality of regions of reduced adhesion, each region of reduced adhesion being disposed adjacent to one of the plurality of pull tab regions.

In examples, the interface patch has a polygonal shape, with sides and corners. In examples, the interface patch has a triangular shape with three sides and three corners.

In examples, the pull tab region is formed at a corner of the interface patch. In examples, the interface patch comprises a plurality of regions of reduced adhesion, each region of reduced adhesion being disposed adjacent to a corner of the interface patch, and wherein a plurality of pull tab regions are formed at the respective corners. The interface patch may comprises one or more indications on the distal side to indicate the location of a pull tab region.

In examples, the interface patch further comprises an aperture for the needle of the subcutaneous medical device. In examples, the interface patch comprises a non-adhesive region surrounding the aperture.

In examples, the interface patch comprises: a first portion comprising the adhesive region, wherein the subcutaneous medical device can be adhered to the first patch portion, and a second portion extending from the first portion and foldable to cover the subcutaneous medical device when the subcutaneous medical device is adhered to the first portion. In examples, the first portion is joined to the second portion at a neck.

In examples, the first portion has a polygonal shape, for example a triangular shape.

In examples, the second portion is smaller than the first portion.

In examples, the second portion comprises an adhesive for adhering to the first portion when the second portion is folded over the first portion.

According to the invention, there is also provided a subcutaneous medical device system for attaching to a patient's skin, comprising a subcutaneous medical device comprising an adhesive surface and a needle for piercing a patient's skin, and an interface patch as described above.

In examples, the subcutaneous medical device comprises a device body and an adhesive patch for adhering the device body to the interface patch.

In examples, the subcutaneous medical device comprises a glucose sensor, for example a flash glucose sensor or a continuous glucose monitor.

In examples, the subcutaneous medical device comprises an infusion set for delivery of a medicament to the patient.

According to the invention, there is also provided method of using the interface patch for attaching the subcutaneous medical device to a patient's skin. The method comprises: applying the interface patch to the patient's skin, attaching the subcutaneous medical device to the interface patch; and removing the interface patch from the patient's skin using the pull tab region.

### BRIEF DESCRIPTION OF THE DRAWINGS

Embodiments of the invention are now described, by way of example only, hereinafter with reference to the accompanying drawings, in which:

FIG. 1 illustrates: (a) a subcutaneous medical device system attachable to a patient and an interface patch, (b) a subcutaneous glucose sensor device and an interface patch, (c) a subcutaneous medical device system including a subcutaneous infusion set device and an interface patch, and (d) an applicator for applying a subcutaneous medical device to a patient;

FIG. 2 illustrates an example interface patch (a) from the proximal side, (b) from the distal side, (c) attached to the skin with the proximal side facing the skin, (d) in a schematic view, and an interface patch having an adhesive provided with a (e) first pattern, (f) second pattern and (g) third pattern;

FIG. 3 illustrates an example interface patch (a) from the distal side, (b) from the proximal side, (c) from the proximal side with a backing film portion removed, (d) initially adhered to the skin, (e) adhered to the skin, and (f) with a subcutaneous medical device applied;

FIG. 4 illustrates an example interface patch (a) in a schematic view, (b) from the distal side, showing a removable release layer on the proximal side, (c) attached to the skin with the proximal side facing the skin, (d) attached to an example subcutaneous medical device to form a subcutaneous medical device system with a cover to be attached to the system and (e) attached to an example subcutaneous medical device to form a subcutaneous medical device system with the cover attached;

FIG. 5 illustrates an example interface patch (a) from the distal side, with a support for aligning an applicator, (b) from the proximal side, (c) from the distal side, attached to a subcutaneous medical device to form a subcutaneous medical device system, with the support attached and (d) attached to a subcutaneous medical device to form a subcutaneous medical device system, with the support removed;

FIG. 6 illustrates an example interface patch (a) in a schematic view, (b) from the distal side, showing a removable release layer, (c) with the removable release layer removed, (d) attached to an example applicator, (e) with a backing layer removed to attach the interface patch to the skin, attached to a subcutaneous medical device to form a subcutaneous medical device system, (f) attached to the skin with a subcutaneous medical device attached, further with the release layers on the proximal side removed and (g) with a cover attached on the distal side to cover the subcutaneous medical device;

FIG. 7 illustrates (a) an example interface patch with a support structure attached, (b) the example interface patch and support structure, with an applicator attached to the support structure, (c) an assembly including the support structure, applicator and interface patch, viewed from the proximal side, (d) the applicator, support structure and interface patch assembly attached to the skin, with the applicator in a first rest position, (e) the applicator, support structure and interface patch assembly attached to the skin, with the applicator in a second actuated position to apply the subcutaneous medical device, (f) a subcutaneous medical device system with the applicator and support structure removed from the assembly, (g) a subcutaneous medical device system including the interface patch and the subcutaneous medical device, with the support structure attached, and (h) the subcutaneous medical device system with the support structure partially removed;

FIG. 8 illustrates an example interface patch (a) with a support structure attached to the interface patch, (b) from the distal side with a support structure attached to the interface patch and including an applicator attached to the support structure, (c) from the proximal side with a support structure attached to the interface patch and including an applicator attached to the support structure and (d) attached to a subcutaneous medical device to form a subcutaneous medical device system;

FIG. 9 illustrates an interface patch having a first patch portion and a second patch portion (a) showing the distal side, (b) showing the proximal side, (c) applied to the patient's skin, (d) applied to the patient's skin and including a subcutaneous medical device, and (e) in a folded-over configuration; and

FIG. 10 illustrates (a) a first example of an interface patch with a region of reduced adhesion, (b) a second example of an interface patch with a region of reduced adhesion, and (c) a third example of an interface patch with a region of reduced adhesion.

### DETAILED DESCRIPTION

Certain terminology is used in the following description for convenience only and is not limiting. The words ‘right’, ‘left’, ‘lower’, ‘upper’, ‘front’, ‘rear’, ‘upward’, ‘down’ and ‘downward’ designate directions in the drawings to which reference is made and are with respect to the described component when assembled and mounted. The words ‘inner’, ‘inwardly’ and ‘outer’, ‘outwardly’ refer to directions toward and away from, respectively, a designated centreline or a geometric centre of an element being described (e.g. central axis), the particular meaning being readily apparent from the context of the description.

Further, as used herein, the terms ‘connected’, ‘attached’, ‘coupled’, ‘mounted’ are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, “first”, “second”, “third” etc. merely indicate that different instances of like objects are being referred to and are not intended to imply that the objects so described must be in a given sequence, either temporally, spatially, in ranking or in any other manner.

Like reference numerals are used to depict like features throughout.

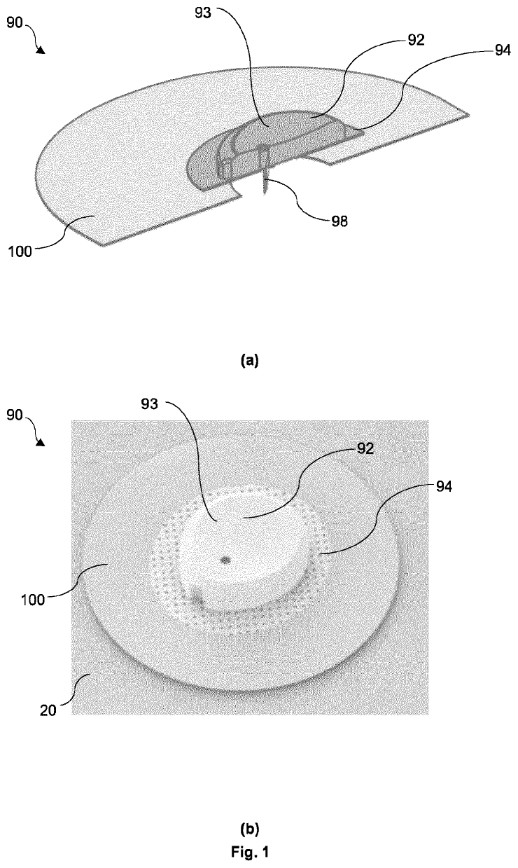

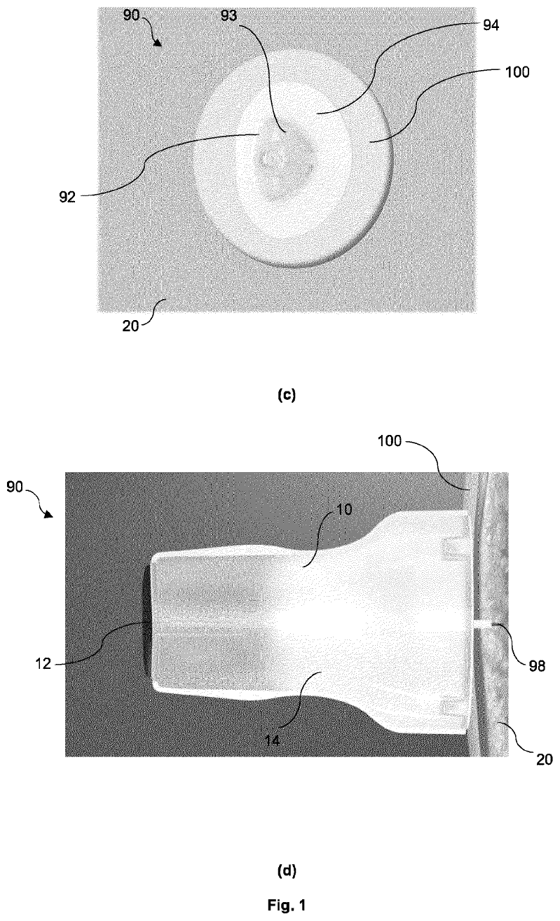

FIGS. 1(a) to 1(c) illustrate example subcutaneous medical device systems 90 that each include a subcutaneous medical device 92. In FIG. 1(b) the subcutaneous medical device 92 is a subcutaneous glucose sensor, and in FIG. 1(c) the subcutaneous medical device 92 is a subcutaneous infusion set of an infusion system. As shown in FIGS. 1(a) to 1(c), in each example the subcutaneous medical device 92 includes a device body 93, an adhesive patch 94 for attaching the device body 93 to a patient's skin 20, and a needle 98 extending from the device body 93 to pierce the patient's skin 20 so that the needle 98 is subcutaneously positioned. The device body 93 is typically rigid and houses other components. The adhesive patch 94 is typically flexible to conform to the skin shape at the attachment site. The adhesive patch 94 has an adhesive surface that faces the skin for attaching the device body 93 to a patient's skin 20, but in other examples the device body 93 itself may have an adhesive surface.

In the example of FIG. 1(b), the subcutaneous glucose sensor 92 includes a sensor adapted to sense a glucose content of blood accessed through the needle 98. The subcutaneous glucose sensor 92 also includes a communications device, such as a communications port or transmitter, for communicating the detected glucose content to an external device. The subcutaneous glucose sensor 92 may be a flash glucose sensor or a continuous glucose monitor.

In the example of FIG. 1(c) the subcutaneous infusion set 92 includes a connector for connecting the needle 98 with an external source of medicament, for example an insulin pump. The subcutaneous infusion set 92 can be used to provide insulin to the patient, through the needle 98, in an intermittent or continuous manner.

FIG. 1(d) illustrates an applicator 10 of the subcutaneous medical device system 90. The applicator 10 is used to apply the subcutaneous medical device 92 to the patient's skin 20. The applicator 10 includes a device body 14 and an actuator 12, in this example a button-operated actuator. In use, the actuator 12 presses the subcutaneous medical device 92 against the patient so that the needle 98 is subcutaneously positioned and the adhesive patch 94 attaches the subcutaneous medical device 92 to the patient. After the subcutaneous medical device 92 has been attached to the patient the applicator 10 is removed.

In other examples, the applicator 10 may have two parts that are collapsed relative to each other to actuate the applicator 10 and press the subcutaneous medical device 10 against the patient.

In various examples, the applicator 10 may be an applicator of the Neria Guard™ system marketed by Convatec™, or the Mio or Mio Advance™ marketed by Medtronic™, or the Autosoft system marketed by Tandem™.

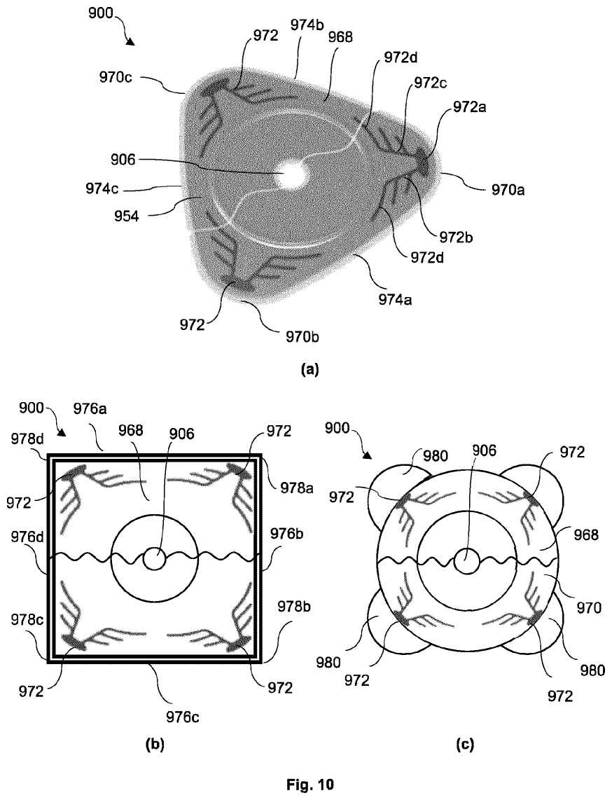

As shown in FIGS. 1(a) to 1(d), the subcutaneous medical device systems 90 include an interface patch 100 disposed between the subcutaneous medical device 92 and the patient's skin 20. In particular, the interface patch 100 is adhered to the patient's skin 20, and the subcutaneous medical device 92 is adhered to the interface patch 100. The interface patch 100 can advantageously improve attachment of the subcutaneous medical device 92 to the patient's skin 20 by providing a better adhesion surface for the adhesive patch 94 than the skin 20. In particular, the interface patch 100 provides a larger surface area in contact with the skin 20 for adhesion. Moreover, the interface patch 100 can reduce skin irritation and damage on removal of the subcutaneous medical device system 90 from the patient. In particular, the larger adhesive surface area provided by the interface patch 100 can use a less strong adhesive, or a pattern-applied adhesive, to make removal of the interface patch 100 from the skin 20 easier without reducing the overall attachment force for the subcutaneous medical device 92. Examples of this are described with reference to FIGS. 10(a) to 10(c).

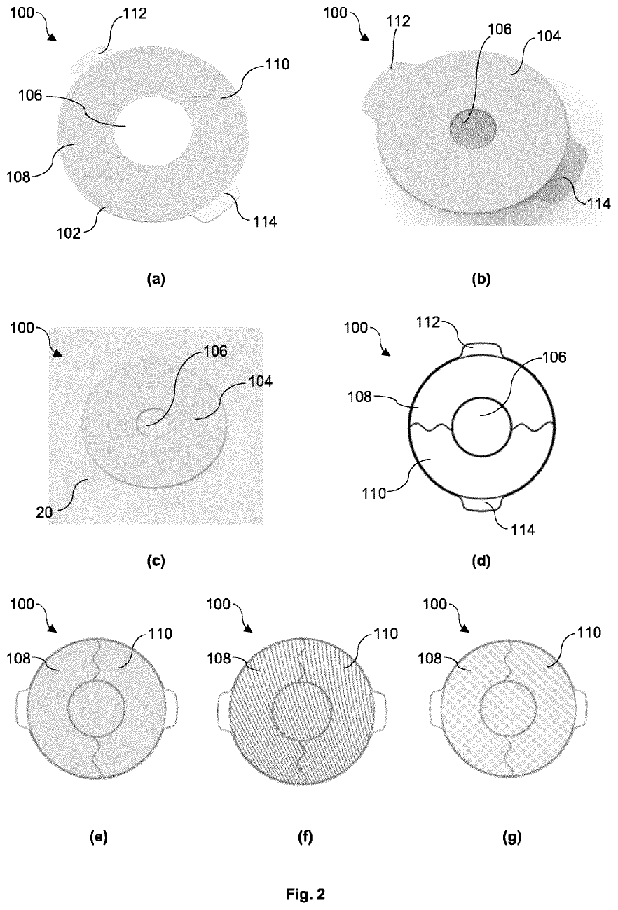

FIG. 2 shows an example interface patch 100 for use with the subcutaneous medical device 92 described above, which may be a glucose monitor or an infusion set of an infusion system as described above. The interface patch 100 has a proximal side 102 as best shown in FIG. 2(a) and FIG. 2(d), and a distal side 104 as best shown in FIG. 2(b). The proximal side 102 includes an adhesive region. The adhesive region extends to the peripheral edge of the interface patch 100. In this example the adhesive region covers the entire proximal side 102 of the interface patch 100. The interface patch 100 in this example has a generally annular shape with an aperture 106. In this example the aperture 106 is positioned in the middle of the interface patch 100, but in other examples the aperture 106 may be offset from the middle of the interface patch 100.

In other examples, not illustrated, the interface patch 100 may include a pierceable region instead of an aperture. The pierceable region is pierceable by the needle 98 of the subcutaneous medical device 92. The pierceable region is positioned in the same location as illustrated for the aperture 106, for example in the centre of the interface patch 100. The pierceable region may be defined by a thinned section of material, or by a region formed of a different material to the rest of the interface patch 100.

As shown, the proximal side 102 of the interface patch 100 is provided with a first release layer 108 on one side (i.e. the left side) and a second release layer 110 on the other side (i.e. the right side) which together cover the adhesive region of the proximal side 102. In this example, the first release layer 108 is integrally formed with a release tab 112 that protrudes beyond the edge of the interface patch 100. Similarly, the second release layer 110 is integrally formed with a release tab 114 that protrudes beyond the edge of the interface patch 100, in a direction opposite the release tab 112. The release layers 108, 110 are removable by lifting the tabs 112, 114 to reveal the adhesive region for adhering the proximal side 102 of the interface patch 100 to the skin 20 of a patient. Although the adhesive region in this example is covered by the first and second release layers 108, 110 that are placed on opposing sides of the proximal side 102 of the interface patch 100, other arrangements are envisaged, such as providing a single release layer to cover the adhesive region.

FIG. 2(c) shows the interface patch 100 with the release layers 108, 110 removed. By removing the release layers 108, 110, the adhesive region on the proximal side 102 of the interface patch 100 is exposed, allowing the interface patch 100 to be adhered to the skin 20, with the proximal side 102 adhered to the skin 20 and the distal side 104 facing away from the skin 20. During use, the aperture 106 of the interface patch 100 allows the needle 98 of the subcutaneous device 92 to pierce the skin 20 underneath the interface patch 100. In some examples, however, the interface patch 100 may be provided without an aperture 106 in which case the needle 98 of the subcutaneous device 92 may pierce through the interface patch 100 directly and into the patient's skin 20.

The interface patch 100 is made from a flexible material, for example any material suitable for use as a medical patch, such as medical tapes. In examples, the interface patch 100 may comprise polyethylene, vinyl, foam, woven or non-woven acrylic, woven or non-woven polyester, urethanes, or rayon acetate woven fabric. The adhesive may comprise a medical adhesive, for example a pressure-sensitive adhesive, such as pressure-sensitive silicone or hydrocolloid. The adhesive may comprise acrylic.

FIGS. 2(a) to 2(d) illustrate a method of using the interface patch 100 as described above. Firstly, the release layers 108, 110 are removed to expose the adhesive region on the proximal side 102 of the interface patch 100. The release layers 108, 110 may be removed by pulling on the release tabs 112, 114 respectively. As shown in FIG. 2(c), after the removal of the release layers 108, 110, the proximal side 102 of the interface patch 100 with the adhesive region is stuck down onto the patient's skin 20. The subcutaneous medical device 92 is then applied to the distal side 104 of the interface patch 100 such that the needle 98 of the subcutaneous medical device 92 aligns with the aperture 106 of the interface patch 100 and the adhesive patch 94 of the subcutaneous medical device 92 adheres around the aperture 106 to secure the subcutaneous medical device 92 to the interface patch 100. By providing an interface patch 100, the subcutaneous medical device 92 is applied to the interface patch 100 instead of directly to the patient's skin 20, using an applicator as previously described.

Although in this example, it is described that both release layers 108, 110 are removed before the interface patch 100 is adhered to the skin 20, the first release layer 108 may be removed to expose half of the adhesive region on the proximal side 102 of the interface patch 100, and then the adhesive region of the interface patch 100 may first be adhered to the skin, before the second release layer 110 is subsequently removed to expose the remaining half of the adhesive region on the proximal side 102, to adhere the rest of the interface patch 100 to the skin 20. This may allow the patient to gradually apply the interface patch 100 to avoid wrinkles or folds in the interface patch 100.

Referring now to FIGS. 2(e) to 2(g), there are shown different patterns for the adhesive region on the proximal side 102 of the interface patch 100. As shown in FIG. 2(e), adhesive may fully cover the proximal side 102 of the interface patch 100 such than when any part of the proximal side 102 is exposed by removal of the release layers 108, 110, the exposed part of the interface patch 100 having adhesive can be adhered to the skin 200. Alternatively, as shown in FIGS. 2(f) and 2(g), the adhesive may be pattern-applied. The adhesive may be pattern-applied to alter the adhesiveness of the proximal side 102 when attached to the patient's skin 20. When the interface patch 100 is positioned between the skin 20 and the subcutaneous medical device 92, the interface patch 100 is larger than the adhesive patch 94 of the subcutaneous medical device 92. Therefore, the interface patch 100 provides a larger surface area for adhesion of the subcutaneous medical device 92 to the skin 20. The adhesion force on the skin 20 is therefore less concentrated in comparison to adhering the adhesive patch 94 of the subcutaneous medical device 92 directly onto the skin. As a result, the adhesive region may be pattern-applied on the proximal side 102 of the interface patch 100 to reduce damage to the skin 20 when the interface patch 100 is removed from the skin 20. The pattern-applied adhesive may be in the form of lines as shown in FIG. 2(f), or the pattern may in the form of a grid as shown in FIG. 2(g), for example, but other patterns are also envisaged.

In an alternative example, the adhesive region on the proximal side 102 of the interface patch 100 may be a less strong adhesive than used on the subcutaneous medical device 92, in particular on the adhesive patch 94. As the interface patch 100 has a larger surface area in contact with the skin than the adhesive patch 94, the adhesive can be weaker while providing the same overall adhesion. This may reduce skin irritation and damage on removal of the subcutaneous medical device system from the patient.

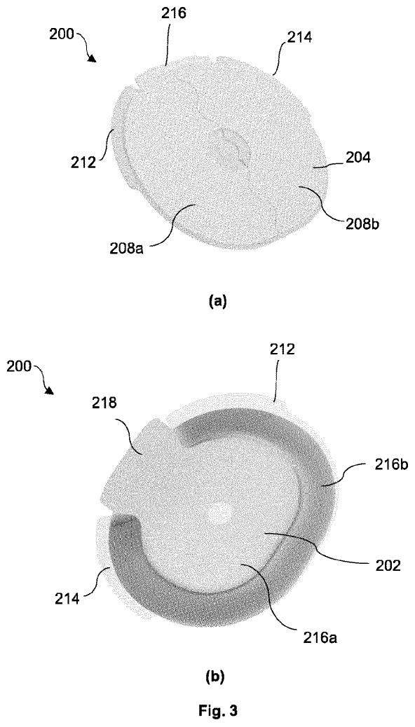

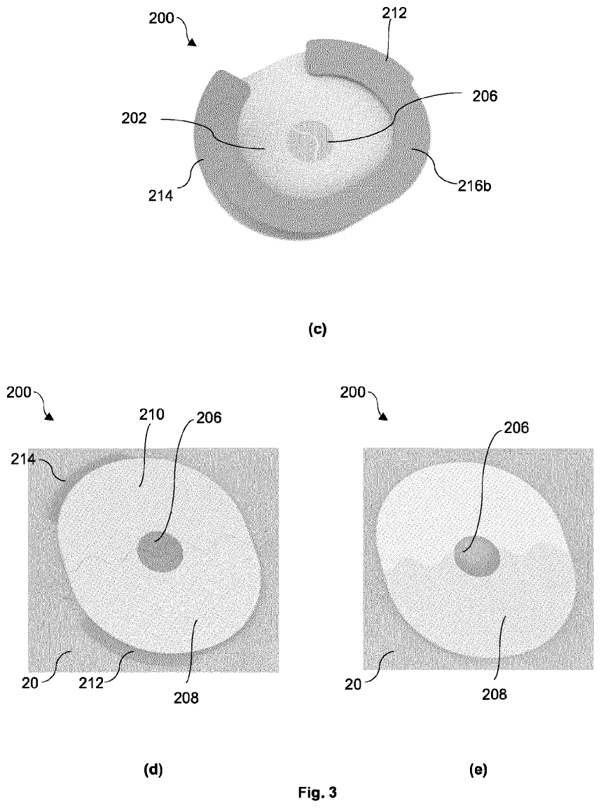

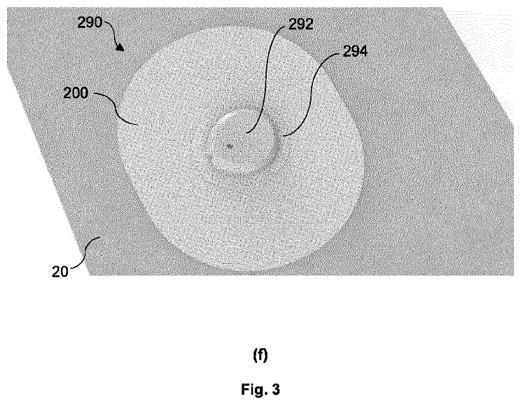

FIG. 3 shows another example interface patch 200 for use with a subcutaneous medical device 292, which in this example is a glucose monitor 292 but may be used with an infusion set as described above. FIG. 3(a) shows the distal side 204 of the interface patch 200 and FIG. 3(b) shows the proximal side 202 of the interface patch 200.

The interface patch 200 is made from a flexible material, for example any material suitable for use as a medical patch, such as medical tapes. In examples, the interface patch 200 may comprise polyethylene, vinyl, foam, woven or non-woven acrylic, woven or non-woven polyester, urethanes, or rayon acetate woven fabric.

As shown in FIG. 3(a), the distal side 204 of the interface patch 200 is provided with a support layer 208. The support layer 208 has a first part 208a on one side and a second part 208b on the other side. The support layer 208 may be adhered to the distal side 204 of the interface patch 200 with a single-use non-tacky adhesive. Alternatively, the support layer 208 may be adhered to the interface patch 200 using an adhesive configured to be removed with the support layer 208. The support layer 208 is provided to increase the rigidity of the interface patch 200 before and during application to the patient so that the patient is better able to avoid wrinkles and folds in the interface patch 200. As explained further below, the support layer 208 is removable from the interface patch 200 after application to the skin.

In a further example illustrated in FIG. 3(b), the proximal side 202 is provided with a backing film 216 having two sections 216a, 216b that are removable to expose the adhesive region on the proximal side 202. The first backing film section 216a is a central section covering the central region of the interface patch 200. The first backing film section 216a includes a pull tab 218 that extends beyond an edge of the interface patch 200 as shown in FIGS. 3(a) and 3(b). The second backing film section 216b covers a peripheral region of the interface patch 200 about a majority of the edge of the interface patch 200, as shown in FIG. 3(b). The first film section 216a and the second film section 216b are independently removable from the interface patch 200 as described further below. The adhesive region may comprise a medical adhesive, for example a pressure-sensitive adhesive, such as pressure-sensitive silicone or hydrocolloid. The adhesive may comprise acrylic.

FIGS. 3(c) to 3(e) illustrate a method of attaching the interface patch 200 to the skin 20. As shown in FIG. 3(c), the first backing film section 216a is removed using pull tab 218 to expose the central part of the proximal side 202 of the interface patch, including the aperture 206. The interface patch 200 is then adhered to the skin 20 at the desired location, as shown in FIG. 3(d). During positioning of the interface patch 200 on the skin the patient can easily handle the interface patch using the edge region still covered by the second backing film portion 216b. Additionally, if provided, the support layer 208 on the distal side 204 of the interface patch 200 will help to prevent wrinkling or folding as the interface patch 200 is applied. Once the central part of the interface patch 200 has been applied the second backing film portion 216b can be removed by pulling on one or both of pull tabs 212 and 214. The entire interface patch 200 is then adhered to the skin 20, as shown in FIG. 3(e).

Once the interface patch 200 has been adhered to the skin 20 the support layer 208 (if provided) can be removed from the distal side 204 of the interface patch 200, exposing the aperture 206 as shown in FIG. 3(e). The support layer 208 thereby helps to maintain the shape and structure of the interface patch 200 as it is adhered to the skin 20, in order to prevent the skin 20 from stretching when the interface patch 200 is applied.

As shown in FIG. 3(f), the subcutaneous medical device 292 can then be applied to the interface patch 200 using the adhesive patch 294 to adhere to the distal side 204 of the interface patch 200. The subcutaneous medical device 292 can be applied using an applicator 10 as described with reference to FIG. 1(d).

By first attaching the interface patch 200 to the skin 20 before applying the subcutaneous medical device 292, the subcutaneous medical device 292 is applied to the interface patch 200 instead of directly to the patient's skin 20, which can help to reduce skin irritation and damage. The interface patch 200 has a large area to ensure adhesion to the skin 20, while reducing concentrated areas of adhesion that could otherwise damage the skin.

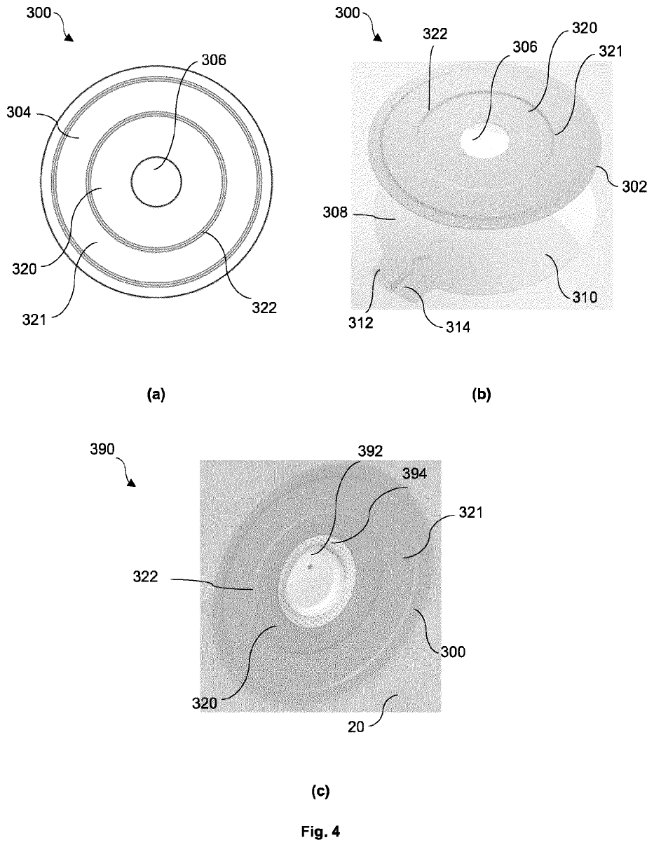

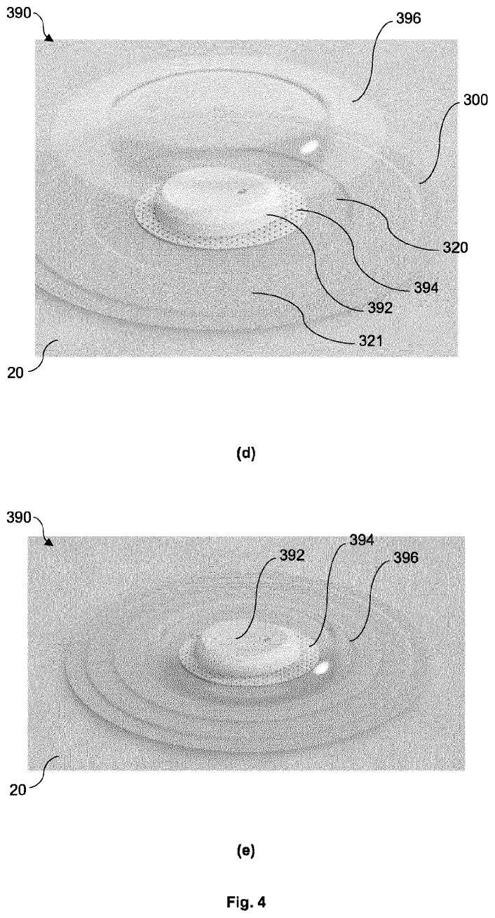

FIG. 4 shows a further example interface patch 300. The interface patch 300 has a proximal side 302 and a distal side 304 opposite the proximal side 302. The proximal side 302 includes an adhesive region. In this example, the interface patch 300 is annular in shape, having a circular outer profile and an aperture 306 in the middle. The interface patch 300 is made from a flexible material, for example any material suitable for use as a medical patch, such as medical tapes. In examples, the interface patch 300 may comprise polyethylene, vinyl, foam, woven or non-woven acrylic, woven or non-woven polyester, urethanes, or rayon acetate woven fabric. The adhesive of the adhesive region may comprise a medical adhesive, for example a pressure-sensitive adhesive, such as pressure-sensitive silicone or hydrocolloid. The adhesive may comprise acrylic.

As shown in FIG. 4(b), the proximal side 302 is provided with first and second release layers 308, 310 that cover an adhesive region on the proximal side 302. The release layers 308, 310 are removable to expose the adhesive region to attach the proximal side 302 of the interface patch 300 to the patient's skin 20. As shown in FIG. 4(b), the first release layer 308 is integrally formed with a release tab 312 that protrudes outward. The second release layer 310 is integrally formed with another release tab 314 that protrudes outward and is adjacent the release tab 312. The release tabs 312, 314 may be pulled or peeled away to remove the respective release layer 308, 310 from the proximal side 302 of the interface patch 300 to expose the adhesive region for sticking the interface patch 300 onto the skin 20. In some examples, the interface patch 300 may have a single part release layer that covers the proximal side 302 of the interface patch 300.

In this example, as shown in FIGS. 4(a) to 4(c), the distal side 304 of the interface patch 300 is provided with a locating edge 322. In particular, the distal side 304 has a region with a raised surface 321 inward of the edge of the interface patch 300. The raised surface 321 provides a relative height difference across different regions of the interface patch 300. The distal side 304 has a recessed surface 320 inward of the raised surface 321 towards the aperture 306. The locating edge 322 is defined at the boundary of the raised surface 321 and the recessed surface 320. Thus, the distal side 304 of the interface patch 300 has a locating edge 322 around the aperture 306 and a circular raised portion 321 outward of the recessed surface 320 towards the edge of the interface patch 300. The locating edge 322 can be used to align the applicator (10, see FIG. 1(d)), in particular the needle (98, see FIG. 1(a)) of the subcutaneous medical device 392 with the aperture 306 when applying the subcutaneous device 392 to the interface patch 300. In particular, a circular part of the applicator can be received against the recessed surface 320, and possibly against the locating edge 322, to align the subcutaneous medical device 392 with the aperture 306. In this example the locating edge 322 is circular, but in various examples it will be appreciated that the shape and size of the locating edge 322, recessed surface 320 and raised surface 321 can be adapted to match different applicators.

As shown in FIGS. 4(d) and 4(e), a cover 396 may optionally be provided to place over the subcutaneous medical device 392 to protect the device 392 from knocking or contamination. In this example, the cover 396 is transparent to view the subcutaneous medical device 392. The cover 396 may be adhered to the interface patch 300, and in particular to the raised surface 321 as shown in FIGS. 4(d) and 4(e). Preferably, the cover 396 is rigid. The cover 396 may comprise any one or more of polyethylene, polypropylene, polyvinyl alcohol, polyurethane, or a thermoplastic elastomer, for example.

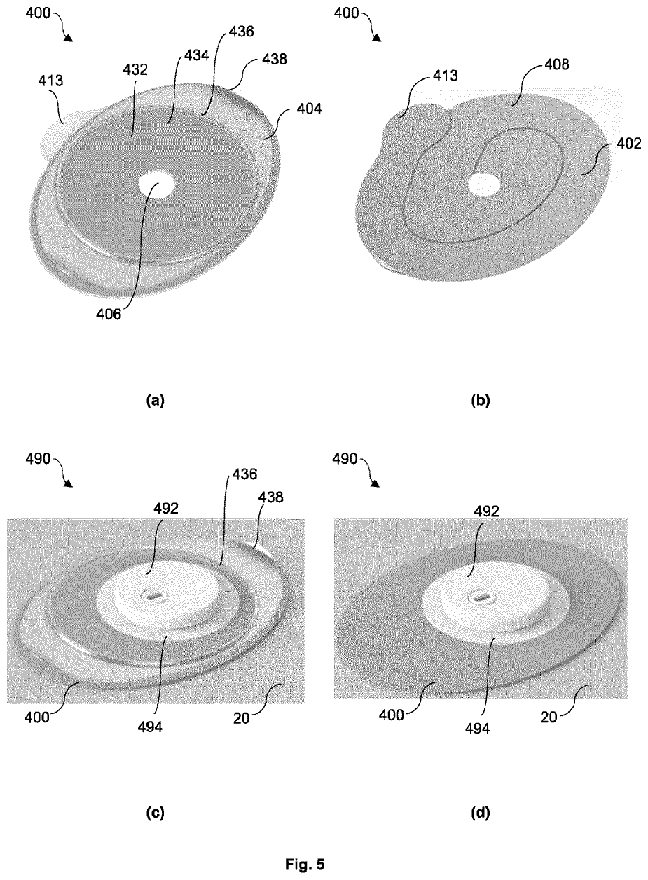

FIG. 5 shows an alternative example of an interface patch 400 that facilitates the alignment of an applicator relative to the interface patch 400. As shown in FIG. 5(b), the interface patch 400 has a proximal side 402. As shown in FIG. 5(a), the interface patch 400 has a distal side 404 opposite the proximal side 402. The proximal side 402 includes an adhesive region. The interface patch 400 is made from a flexible material, for example any material suitable for use as a medical patch, such as medical tapes. In examples, the interface patch 400 may comprise polyethylene, vinyl, foam, woven or non-woven acrylic, woven or non-woven polyester, urethanes, or rayon acetate woven fabric. The adhesive of the adhesive region may comprise a medical adhesive, for example a pressure-sensitive adhesive, such as pressure-sensitive silicone or hydrocolloid. The adhesive may comprise acrylic.

The proximal side 402 has a release layer 408 that is provided to cover an adhesive region on the interface patch 400. In this example, the release layer 408 has a spiral profile, but it will be appreciated that the release layer 408 may have alternative shapes and configurations, for example a single sheet or two parts as shown in FIG. 2. At the end of the release layer 408, there is provided a pull tab 413 which extends outward of the proximal side 402. The pull tab 413 can be used to remove the release layer 408 to expose the adhesive region on the proximal side 402. The interface patch 400 can then be adhered to the skin 20 by the adhesive on the proximal side 402 of the interface patch 400.

As best shown in FIG. 5(a), a support 432 is provided on the distal side 404 of the interface patch 400. The support 432 is preferably rigid. The support 432 is attached, for example adhered, to the distal side 404 of the interface patch 400. The support 432 holds the interface patch 400 in a flat position during application of the interface patch 400 to the user's skin, helping to avoid wrinkles and folds in the interface patch 400.

In addition, as illustrated in FIGS. 5(a) and 5(c), the support 432 includes an opening 434 having an edge 436. The opening 434 surrounds the aperture 406 in the interface patch 400 and allows application of a subcutaneous medical device 492 to the interface patch 400 through the opening 434 using an applicator as described with reference to FIG. 1(d). The edge 436 may serve as a locating edge for the applicator to ensure alignment of the applicator and the aperture 406 so that the needle of the subcutaneous medical device is aligned with the aperture 406. In this example the opening 434 and edge 436 are circular, but it will be appreciated that other shapes and sizes may be provided for different applicators. The edge 436 may be formed by the thickness of the support 432, or it may include a lip or protrusion.

As shown in FIG. 5(d), after application of the subcutaneous medical device 492 the support 432 can be removed, for example by pulling on the tab 438 shown in FIGS. 5(a) and 5(c). The support 432 can be adhered to the interface patch 400 using a single-use, non-tacky adhesive that offers a low adhesion force, so the support 432 can be separated from the interface patch 400 without disturbing the adhesion of the interface patch 400 to the skin 20. Alternatively, the support 432 can be adhered to the interface patch 400 using an adhesive that remains on the support 432 when the support 432 is removed from the interface patch 400.

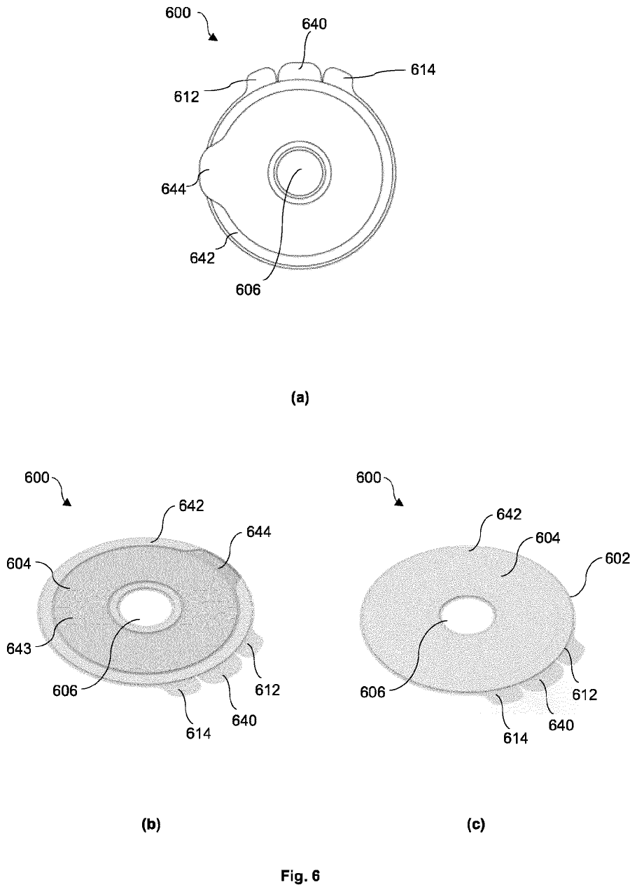

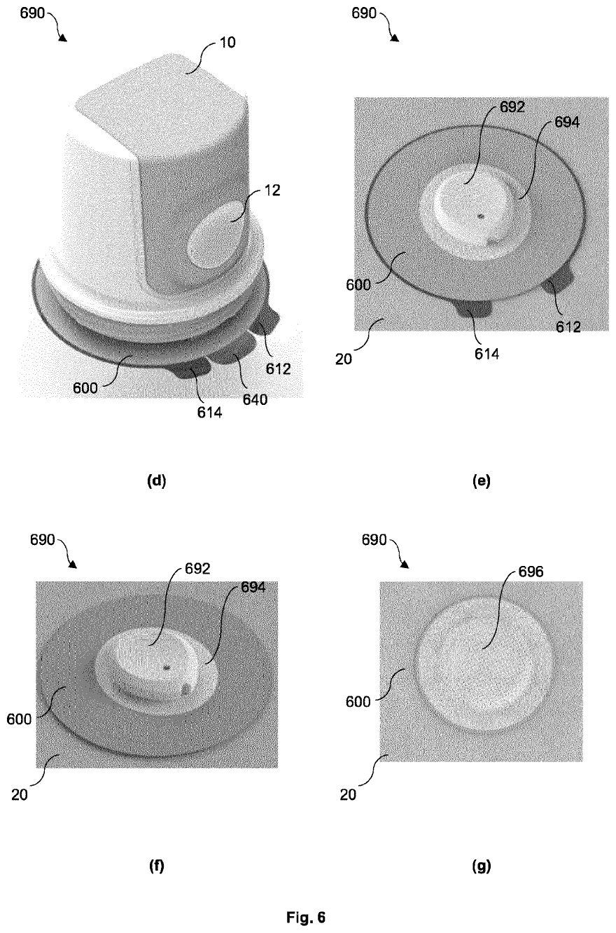

FIG. 6 shows a further example interface patch 600. In this example the interface patch 600 can be mounted to the applicator 10 before the interface patch 600 is attached to the patient's skin 20. The interface patch 600 includes a distal side 604 as shown in FIGS. 6(a), 6(b) and 6(c), and a proximal side 602 on the opposite side. In this example, the proximal side 602 is provided with an adhesive region for adhering the interface patch 600 to the patient's skin. The interface patch 600 is made from a flexible material, for example any material suitable for use as a medical patch, such as medical tapes. In examples, the interface patch 600 may comprise polyethylene, vinyl, foam, woven or non-woven acrylic, woven or non-woven polyester, urethanes, or rayon acetate woven fabric. The adhesive of the adhesive region may comprise a medical adhesive, for example a pressure-sensitive adhesive, such as pressure-sensitive silicone or hydrocolloid. The adhesive may comprise acrylic.

The adhesive region on the proximal side 602 is covered by a release layer (not shown) that may be the same as described with reference to FIG. 3. In particular, the release layer may have a central portion with a pull tab 640, and a peripheral portion with pull tabs 612, 614. The release layer on the proximal side 602 can be removed by pulling the pull tabs 640, 612, 614. However, it will be appreciated that various release layers may be provided, including a single sheet or two parts as shown in FIG. 2.

A central portion of the distal side 604 is provided with an adhesive region. A release layer 643 covers the central adhesive region. The adhesive may comprise a medical adhesive, for example a pressure-sensitive adhesive, such as pressure-sensitive silicone or hydrocolloid. The adhesive may comprise acrylic. The release layer 643 in this example is integrally formed with a central release layer tab 644 that can be pulled to remove the central release layer 643. A peripheral edge 642 of the distal side 604 is adhesive-free.

The method of using the interface patch 600 is best illustrated in FIGS. 6(b) to 6(f). Firstly, the release layer 643 is removed from the distal side 604 by pulling on the release tab 644 to expose an adhesive surface below the release layer 643 on the distal side 604 of the interface patch 600. With the adhesive surface exposed, an applicator 10 is attached to the distal side 604 of the interface patch 600 by the adhesive as shown in FIG. 6(d). The adhesive can adhere to an end face of the applicator 10.

The release tab 640 is then pulled to peel off the central portion of the release layer (not shown) on the proximal side 602 of the interface patch 600. By peeling off the release layer on the proximal side 602, the adhesive region is exposed for attaching the proximal side 602 to the skin 20. The assembly 690, including the applicator 10 and the interface patch 600, can now be attached to the patient's skin 20. Once the proximal side 602 is attached to the skin 20, the applicator 10 is actuated using the actuator 12 to insert the needle of the subcutaneous medical device 692 into the skin 20. The adhesive patch 694 of the subcutaneous medical device 692 adheres to the distal side 604 of the interface patch 600. After the subcutaneous medical device 692 is attached to the interface patch 600, the applicator 10 is removed and the remaining release tabs 612, 614 are pulled to peel off any remaining release layers on the proximal side 602.

As shown in FIG. 6(g), optionally a cover 696 may be provided to protect the device 692 from knocking or contamination. The cover 696 can be adhered to the interface patch 600 using the adhesive on the distal side 604 that was used to attach the applicator 10. The cover 696 may comprise any one or more of polyethylene, polypropylene, polyvinyl alcohol, polyurethane, or a thermoplastic elastomer, for example.

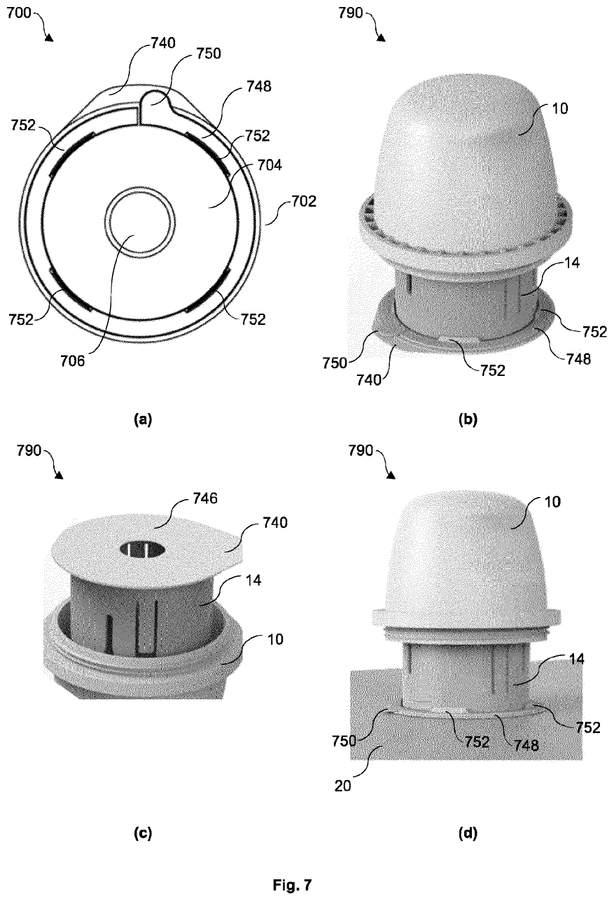

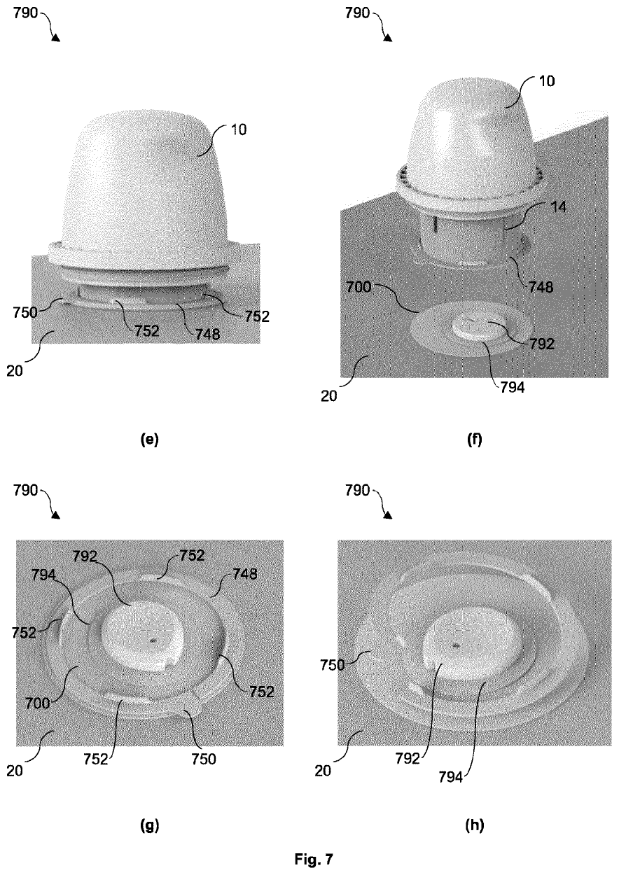

FIG. 7 shows a further example interface patch 700 that is attachable to the applicator 10 before attaching the interface patch 700 to the patient's skin 20. The distal side 704 of the interface patch 700 is shown in FIG. 7(a). The applicator 10 is attachable to the distal side 704 of the interface patch 700 as shown in FIG. 7(b). The proximal side of the interface patch 700 includes an adhesive region for adhering the interface patch 700 to the patient's skin. The interface patch 700 is made from a flexible material, for example any material suitable for use as a medical patch, such as medical tapes. In examples, the interface patch 700 may comprise polyethylene, vinyl, foam, woven or non-woven acrylic, woven or non-woven polyester, urethanes, or rayon acetate woven fabric. The adhesive of the adhesive region may comprise a medical adhesive, for example a pressure-sensitive adhesive, such as pressure-sensitive silicone or hydrocolloid. The adhesive may comprise acrylic.

In this example, the interface patch 700 includes a support structure 748 on the distal side 704 of the interface patch 700 for attaching the applicator 10 to the interface patch 700. The support structure 748 extends along the edge of the interface patch 700. In this example, the support structure 748 is annular in shape. The support structure 748 forms a tape (e.g. a semi-rigid support liner) extending about the edge of the interface patch 700 and includes a tab 750 integrally formed with the support structure 748. The support structure 748 has an outer ring edge which is sized and shape to correspond with the edge of the interface patch 700, and an inner ring edge towards the aperture 706 of the interface patch 700. The support structure 748 is adhered to the distal side 704 of the interface patch 700, preferably with a single-use, non-tacky adhesive. Alternatively, the support structure 748 is adhered to the interface patch 700 using an adhesive that remains on the support structure 748 when the support structure 748 is removed from the interface patch 700. The inner edge of the support structure 748 is provided with four support arms 752 equidistantly spaced around the inner edge that engage with the body 14 of the applicator 10 in use. The support arms 752 act to clip onto the body 14 of the applicator 10, or grip the body 14 of the applicator 10. The supports arms 752 may be resiliently deformable. When the support structure 748 is attached to the applicator 10 the subcutaneous medical device within the applicator 10 is aligned with the aperture 706 in the interface patch 700.

The proximal side 702 of the interface patch 700 is provided with a release layer 746 that covers an adhesive region on the proximal side 702, and includes a release tab 740. The adhesive region on the proximal side 702 is used to adhere the interface patch 700 to the patient's skin,

FIGS. 7(b) to 7(h) illustrate the method of using the interface patch 700. The interface patch 700 and support structure 748 may be provided pre-assembled (with the support structure 748 adhered to the distal side 704 of the interface patch 700), or the patient may adhere the support structure 748 to the interface patch in the position shown in FIG. 7(a). The device body 14 of the applicator 10 is attached to the interface patch 700 using the support structure 748. In this example, the support arms 752 are resiliently biased against the device body 14 to firmly hold the applicator 10 in place. The interface patch 700 and the applicator 10 may be pre-assembled, or the patient may attach the interface patch 700 to the applicator 10 using the support structure 748. As shown in FIG. 7(c), the release layer 746 on the proximal side 702 of the interface patch 700 is removed by pulling on the release tab 740 to peel the release layer 746 off, exposing the adhesive region. The assembly 790 including the applicator 10 and the interface patch 700 is then attached to the skin 20, as shown in FIG. 7(d). As shown in FIG. 7(e), the applicator 10 is used to apply the subcutaneous medical device 792. In this example, the applicator 10 is actuated by pushing the head of the applicator 10 downwards to collapse the device body 14 into the head, but it will be appreciated that other types of applicator 10 may be used, for example button-actuated applicators. The subcutaneous medical device 792 is applied such that the needle of the subcutaneous medical device 792 pierces into the skin 20 of the patient. At the same time, the adhesive patch 794 of the subcutaneous medical device 792 adheres around the aperture 706 to secure the subcutaneous medical device 792 to the interface patch 700.

As shown in FIG. 7(f), the applicator 10 can be removed from the interface patch 700 together with the support structure 748. Alternatively, as shown in FIGS. 7(g) and 7(h), the applicator 10 can be removed on its own from the interface patch 700, and then the support structure 748 can be subsequently removed after the removal of the applicator 10. The tab 750 can be pulled to support the removal of the support structure 748. Thus, the applicator 10 is first assembled with the interface patch 700, before the assembly 790 is attached the skin 20. This removes the need to align the applicator 10 with the interface patch 700, which is particularly beneficial if the interface patch 700 is placed in a location that is hard to reach or see, for example the back of the arm.

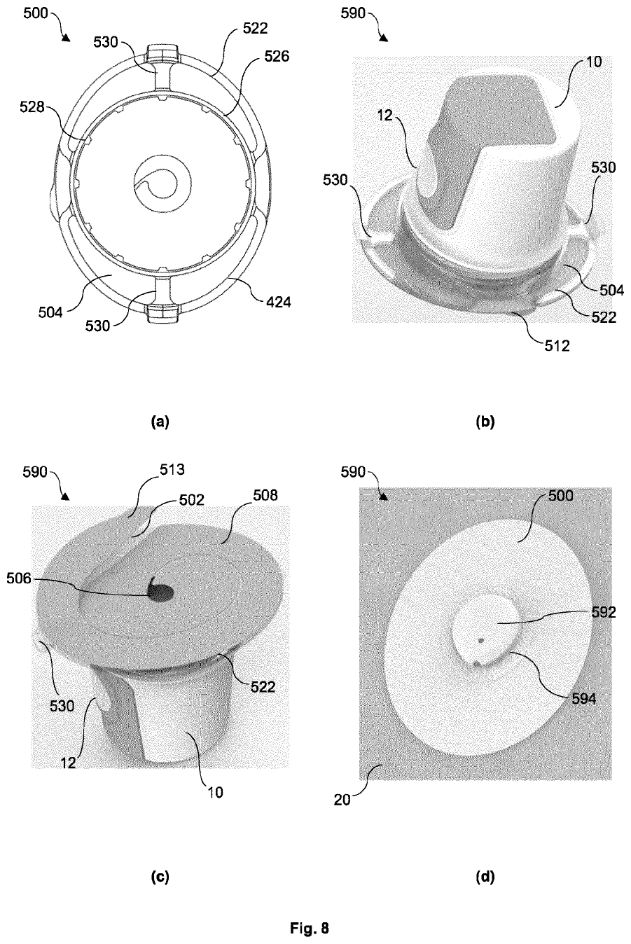

Referring now to FIG. 8, there is shown a further example interface patch 500 that can be attached to an applicator 10 before application to the patient. The interface patch 500 includes a proximal side having an adhesive region for attaching the interface patch 500 to the patient's skin. The applicator 10 is attachable to a distal side of the interface patch 500, opposite to the proximal side. The interface patch 500 is made from a flexible material, for example any material suitable for use as a medical patch, such as medical tapes. In examples, the interface patch 500 may comprise polyethylene, vinyl, foam, woven or non-woven acrylic, woven or non-woven polyester, urethanes, or rayon acetate woven fabric. The adhesive of the adhesive region may comprise a medical adhesive, for example a pressure-sensitive adhesive, such as pressure-sensitive silicone or hydrocolloid. The adhesive may comprise acrylic.

In this example, the interface patch 500 is provided with a support structure 522 which is attachable to the interface patch 500 and also attachable to an applicator 10 for applying the subcutaneous medical device 592.

As shown in FIGS. 8(a) and 8(b), the support structure 522 has a frame 524 sized and shaped corresponding with the interface patch 500. In this example, the interface patch 500 and the frame 524 are elliptical, but it will be appreciated that the interface patch 500 and frame 524 may be differently shaped. The interface patch 500 is attached to one side of the support structure 522, for example by an adhesive (preferably a single-use, non-tacky adhesive). In examples, the support structure 522 is adhered to the interface patch 500 using an adhesive that remains on the support structure 522 when the support structure 522 is removed from the interface patch 500. The support structure 522 is also provided with tabs 530 for peeling the support structure 522 away from the interface patch 500 after application as described below. The support structure 522 includes a circular mount 526 for engaging the applicator 10. In this example the mount 526 is provided with resilient members 528 that protrude inwardly to resiliently engage the applicator 10 and hold the support structure 522 (and interface patch 500) on the applicator 10. In other examples, the mount 526 may be a push fit onto the applicator 10, or may be attached to the applicator 10 using a fastener. When the support structure 522 is attached to the applicator 10 the subcutaneous medical device within the applicator 10 is aligned with the aperture 506 in the interface patch 500.

As shown in FIG. 8(c), the proximal side 502 of the interface patch 500 has an adhesive region that is covered by a release layer 508, which in this example is a single piece release layer having a spiral form. However, it will be appreciated that other shapes of release layer 508 may alternatively be provided.

FIGS. 8(b) to 8(d) illustrate the method of using the interface patch 500. Firstly, the interface patch 500 is attached to the support structure 522. The interface patch 500 and support structure 522 may be provided pre-assembled, or the patient may attach the interface patch 500 to the support structure 522.

Next, the support structure 522 (and interface patch 500) is attached to the applicator 10. In some examples, the patient attaches the support structure 522 to the applicator 10, but in other examples they are provided pre-assembled. To attach the applicator 10 and support structure 522 an end of the applicator 10 is pushed into the mount 526 so that the resilient members 528 engage the applicator 10.

After the applicator 10 is secured in place relative to the interface patch 500, the release layer 508 is removed from the proximal side 502 of the interface patch 500 to expose the adhesive region for securing the assembly 590 onto the patient's skin 20. The assembly 590 is then applied to the skin with the adhesive region on the interface patch 500 being pressed onto the skin.

After the assembly 590, including the interface patch 500, applicator 10 and support structure 522, is attached to the skin 20 by the adhesive region on the proximal side 502 of the interface patch 500, the actuator 12 is operated to insert the needle of the subcutaneous medical device 592 into the skin 20. The subcutaneous medical device 592 is applied in this way to the distal side 504 of the interface patch 500 such that the needle of the subcutaneous medical device 592 aligns with the aperture 506 of the interface patch 500. The adhesive patch 594 of the subcutaneous medical device 592 adheres around the aperture 506 to secure the subcutaneous medical device 592 to the interface patch 500. By attaching the applicator 10 to the interface patch 500 before attachment of the interface patch 500 to the skin 20, the need to align the applicator 10 to the interface patch 500 after attachment is removed. This is particularly advantageous if the interface patch 500 and the subcutaneous medical device 592 need to be attached to the patient's skin 20 at a location that is hard to see or hard to reach, for example the back of the arm.

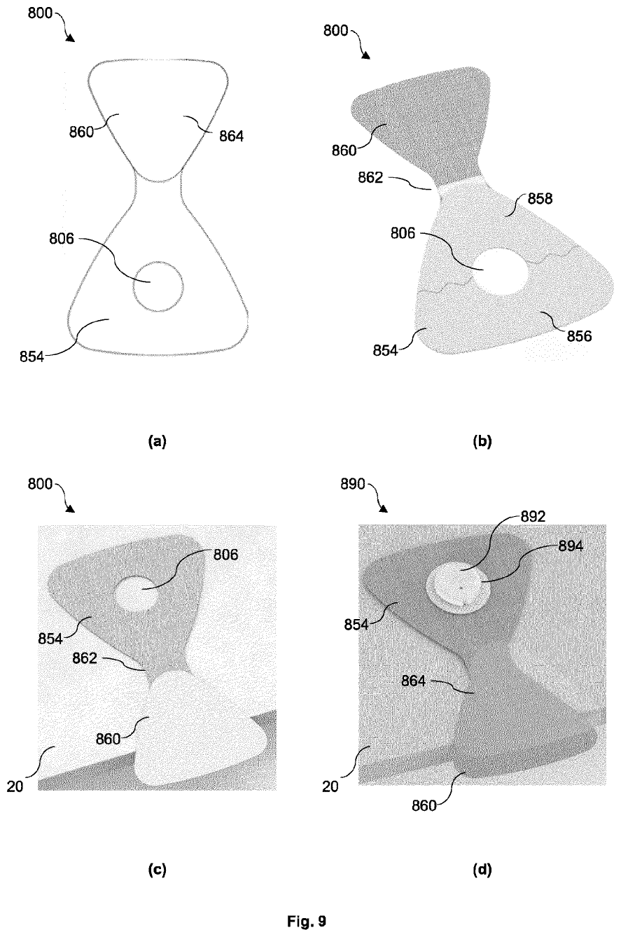

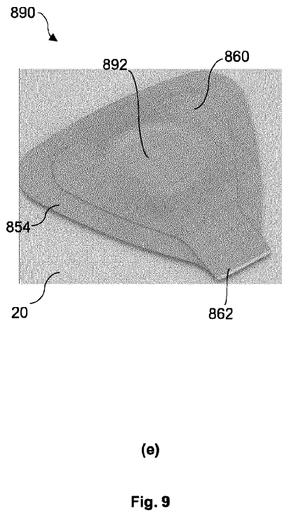

FIGS. 9(a) and 9(b) illustrate an example interface patch 800 for use with the subcutaneous medical device 92 described above. In particular, the example interface patch 800 may be used with a glucose monitor (e.g., a flash glucose monitor or a continuous glucose monitor), or an infusion set of an infusion system, as described above.

As shown, the interface patch includes a first patch portion 854 and a second patch portion 860. The first patch portion 854 and the second patch portion 860 are attached to each other. FIG. 9(a) shows a distal side of the first patch portion 854 and FIG. 9(b) shows a proximal side of the first patch portion 854. In use, the proximal side is adhered to the patient's skin, and the distal side, being opposite to the proximal side, faces away from the patient's skin.

The interface patch 800 is made from a flexible material, for example any material suitable for use as a medical patch, such as medical tapes. In examples, the interface patch 800 may comprise polyethylene, vinyl, foam, woven or non-woven acrylic, woven or non-woven polyester, urethanes, or rayon acetate woven fabric.

As illustrated, in this example the first patch portion 854 has a generally triangular shape, with three major sides and rounded corners joining the major sides. Similarly, the second patch portion 860 has a generally triangular shape, with three major sides and rounded corners joining the major sides. The first patch portion 854 and the second patch portion 860 are joined to each other at a corner area of each of the first patch portion 854 and the second patch portion 860. The corner area therefore forms a neck 862 joining the first patch portion 854 and the second patch portion 860.

Therefore, as illustrated in FIGS. 9(a) and 9(b), before use the triangular shapes of the first patch portion 854 and the second patch portion 860 are arranged in opposite orientations. As will be described below, during use the second patch portion 860 is folded over onto the first patch portion 854, in which position the triangular shapes of the first patch portion 854 and the second patch portion 860 are arranged in the same orientation. The first patch portion 854 is larger than the second patch portion 860, so that when the second patch portion 860 is folded over onto the first patch portion 854 the edges of the second patch portion 860 are inwards of the edges of the first patch portion 854 (see FIG. 9(e)). This prevents the second patch portion 860 from adhering directly to the patient's skin.

In the illustrated example the first patch portion 854 is generally triangular, but in other examples the first patch portion 854 may have an alternative shape, for example a circle or other polygonal shape (e.g., square, pentagonal, hexagonal). The second patch portion 860 may have a shape corresponding to that of the first patch portion 854, or the second patch portion 860 may have a different shape. For example, the first patch portion 854 may be generally triangular, as illustrated, and the second patch portion 860 may be circular.

As illustrated, the first patch portion 854 includes an aperture 806. As will become apparent below, during use the aperture 806 allows the needle of the subcutaneous medical device 892 to directly pierce the patient's skin underneath the interface patch 800. The aperture is centrally located in the first patch portion 854, but may be offset in order to align with the needle of the subcutaneous medical device 892 when the subcutaneous medical device 892 is adhered to the interface patch 800.

In some examples the interface patch 800 does not include the aperture 806, and instead the needle of the subcutaneous medical device 892 can pierce through the first patch portion 854 and the patient's skin in use. In such examples, the interface patch 800 may include a thinner or weaker area that can be more easily pierced by the needle.

The proximal side of the first patch portion 854 includes a first adhesive region. As shown in FIG. 9(b), the first adhesive region is covered by a release layer 856, 858. In this example, the release layer 856, 858 has a first portion 856 and a second portion 858, but it will be appreciated that various arrangements of the release layer 856, 858 are possible, including a single part.

Similarly, the distal side of the second patch portion 860 includes a second adhesive region. As shown in FIG. 9(a), the second adhesive region is covered by a release layer 864. In this example, the release layer 864 is a single part, but it will be appreciated that the release layer 864 may have more than one portion.

The release layer 856, 858 on the first patch portion 854 and the release layer 864 on the second patch portion 864 are removable to expose the first and second adhesive regions, respectively. In particular, a user can peel away the release layers 856, 858, 864.

The adhesive of the first adhesive region may be applied as a uniform layer across the entire first patch portion 854, or as a uniform layer across only a part of the first patch portion 854. In examples, the adhesive of the first adhesive region may be pattern-applied across the entire first patch portion 854, or may be pattern-applied across only a part of the first patch portion 854. The adhesive may comprise a medical adhesive, for example a pressure-sensitive adhesive, such as pressure-sensitive silicone or hydrocolloid. The adhesive may comprise acrylic. Examples of the first adhesive region are described with reference to FIGS. 10(a) to 10(c).

Similarly, the adhesive of the second adhesive region may be applied as a uniform layer across the entire second patch portion 860, or as a uniform layer across only a part of the second patch portion 860. In examples, the adhesive of the second adhesive region may be pattern-applied across the entire second patch portion 860, or may be pattern-applied across only a part of the second patch portion 860. The adhesive may comprise a medical adhesive, for example a pressure-sensitive adhesive, such as pressure-sensitive silicone or hydrocolloid. The adhesive may comprise acrylic.

FIGS. 9(c) to 9(e) illustrate a method of using the interface patch 800 described above. In a first step, as shown in FIG. 9(c), the release layer 856, 858 is removed from the first adhesive region on the first patch portion 854, and the first patch portion 854 is adhered to the patient's skin 20 using the first adhesive region. The interface patch 800 is adhered to the patient at the desired attachment site of the subcutaneous medical device 892 described above. In particular, the aperture 806 in the first patch portion 854, if present, can be aligned with the desired puncture point for the needle of the subcutaneous medical device 892.

Next, as shown in FIG. 9(d), the subcutaneous medical device 892 is applied to the first patch portion 854. In particular, the needle of the subcutaneous medical device 892 is aligned with the aperture 806 and the adhesive patch of the subcutaneous medical device 892 adheres about the aperture 806 to adhere the subcutaneous medical device 892 to the distal side of the interface patch 800. In examples, the subcutaneous medical device 892 is applied using an applicator as described with reference to FIG. 1(d).

Next, as shown in FIG. 9(e), the release layer 864 can be removed from the second adhesive region on the distal side of the second patch portion 860, and the second patch portion 860 can be folded over on top of the first patch portion 854 and over the subcutaneous medical device 892. The interface patch 800 is folded about the neck 862 adjoining the second patch portion 860 and the first patch portion 854. The second adhesive region will adhere the second patch portion 860 to the distal side of the first patch portion 854. The second adhesive region may additionally adhere the second patch portion 860 to the subcutaneous medical device 892, although in some examples the second adhesive region is arranged to only adhere to the first patch portion 854 and not to the subcutaneous medical device 892 (e.g., the second adhesive region extends only about the perimeter of the second patch portion 860).

As illustrated, once folded, the second patch portion 860 covers the subcutaneous medical device 892. This advantageously acts to improve the holding force attaching the subcutaneous medical device 892 to the patient, acts to protect the subcutaneous medical device 892 (e.g., against knocks, water, and dirt), and may make the subcutaneous medical device 892 more discreet.

In examples in which the subcutaneous medical device 892 is an infusion set, a connecting tube may extend between the first and second patch portions 854, 860 to connect with the infusion pump.

In some examples, the interface patch 800 may include a hard or rigid cover arranged to cover the subcutaneous medical device 892 and be retained in position by the second patch portion 860. The cover may be integrated into the second patch portion 860 or may be a separate component positioned over the subcutaneous medical device 892 before the second patch portion 860 is folded over.

FIGS. 10(a) to 10(c) illustrate examples of an interface patch 900 with an adhesive region 968 for adhering the interface patch 900 to a patient's skin. FIGS. 10(a) to 10(c) illustrate the proximal sides of the interface patches 900, which include the adhesive region 968 and which is attached to the patient's skin in use.

The interface patch of FIG. 10(a) is the same as described with reference to FIGS. 9(a) to 9(e). In particular, the interface patch 900 includes a first patch portion 954 that is adhered to the skin, and a second patch portion (not shown in FIG. 10(a)) that can be folded onto the first patch portion 954 after the subcutaneous medical device has been attached to the first patch portion 954 as described above. The first patch portion 954 includes an aperture 906 for the needle of the subcutaneous medical device, as previously described.

FIG. 10(a) illustrates the proximal side of the first patch portion 954 after the second patch portion (not shown in FIG. 10(a)) has been folded over. As illustrated, the first patch portion 954 includes an adhesive region 968 for adhering the interface patch 900 to the patient's skin. A release layer (not illustrated) is provided over the first adhesive region 968 and is removed before use, as described above. The adhesive region 968 extends to the peripheral edge of the first patch portion 954. In the illustrated example the adhesive region 968 is formed about a periphery of the first patch portion 954, and does not extend to the area surrounding the aperture 906. However, in other examples the adhesive region 968 may extend across the entire surface of the first patch portion 958, or only in other areas of the surface of the first patch portion 958.

As with the example of FIGS. 9(a) to 9(e), the first patch portion 954 has a generally triangular shape, with three major sides 974a-974c and three rounded corners 970a-970c joining the major sides 974a-974c.

After use, for example after several days or weeks of a patient wearing the interface patch 900 and subcutaneous medical device, the interface patch 900 is removed from the patient's skin. To do this, the patient will peel the interface patch 900 from the skin. The generally triangular shape of the first patch portion 954 will encourage the patient to peel from one of the corners 970a-970c. Additionally, the interface patch 900 may include some visual indication that the patient should peel from one of the corners 970a-970c. At least one of the corners 970a-970c, and preferably all of the corners 970a-970c, thereby provide a pull tab region for removal of the interface patch 900.

As shown in FIG. 10(a), the first adhesive region 968 includes a region of reduced adhesion 972. In this example there is a region of reduced adhesion 972 provided at each corner 970a-970c—so three regions of reduced adhesion 972. The or each region of reduced adhesion 972 may comprise no adhesive, or a reduced thickness of adhesive, or a different, less strong adhesive. In this way, the region of reduced adhesion 972 is adhered to the patient's skin with reduced strength compared to the remainder of the adhesive region 968.

As illustrated, the region of reduced adhesion 972 includes a first region 972a located at or proximal to the periphery of the first adhesive area 968 at the corner 970a. The first region 972a is therefore disposed at an edge of the first patch portion 954. The region of reduced adhesion 972 also includes a first line 972b and a second line 972c extending away from the first region 972a in a divergent manner, generally parallel to the adjacent major sides 974a, 974b of the first patch portion 954. In this way, when the patient begins peeling the interface patch 900 from their skin during removal, the region of reduced adhesion 972 will reduce the skin tension generated during peeling and thereby reduce skin irritation and damage. The divergent arrangement of the first line 972b and the second line 972c reduce skin tension in the directions of the first and second lines 972b, 972c.

In other examples, the region of reduced adhesion 972 may comprise a single line extending from the first region 972a. The single line may extend towards a centre of the first patch portion 954.

Additionally, as shown, the or each region of reduced adhesion 972 may include one or more arms 972d that extend from the first line 972b and the second line 972c. The arms 972d extend towards the majors sides 974a-974c of the first patch portion 954, but may additionally or alternatively extend in the opposite direction. The arms 972d further reduce skin tension as the interface patch 900 is peeled from the patient's skin by providing breaks as the interface patch 900 is peeled, which releases the skin tension.

As shown in FIG. 10(a), there is a region of reduced adhesion (i.e. tension) 972 for each of the corners 970a-970c of the first patch portion 954. In other examples, the first patch portion 954 may include a pull tab, for example an adhesive-free region (or protrusion, for example). The pull tab may extend from an edge of the first patch portion 954. In such examples, there may be a region of reduced adhesion 972 positioned adjacent to the pull tab.

In other examples, as mentioned above, the first patch portion 954 may have an alternative shape, for example a circle or other polygonal shape (e.g., square, pentagonal, hexagonal). In each example, the first patch portion 954 may include at least one region of reduced adhesion 972 positioned adjacent to a pull tab region (i.e. pulling point) of the first patch portion. The pull tab region may be a corner, as shown in FIG. 10(a), or a pull tab.

In a further example, shown in FIG. 10(b), the interface patch 900 has a generally quadrilateral shape, for example square or rectangular. FIG. 10(b) shows a square interface patch 900 with four sides 976a-976d and four corners 978a-978d. The interface patch 900 includes a central aperture 906 as described above. At least one of the corners, and preferably all of the corners, form pull tab regions for removing the interface patch 900. The adhesive region 968 extends across the entire surface of the interface patch 900, and may be evenly applied or pattern-applied. The adhesive region 968 includes at least one region of reduced adhesion 972. The region of reduced adhesion 972 is located adjacent to a corner 978a and is substantially as described above with reference to FIG. 10(a). In particular, the region of reduced adhesion 972 has a first region 972a and lines 972b, 972c extending from the first region 972a as shown in FIG. 10(a). Preferably the lines 972b, 972c are divergent. Preferably, arms 972 extend from the lines 972b, 972c. In this illustrated example the interface patch 900 includes four regions of reduced adhesion 972—one positioned adjacent to each corner 978a-978d. As described above, the region(s) of reduced adhesion 972 may reduce skin irritation and damage when the interface patch 900 is peeled from the patient's skin.

In a further example, shown in FIG. 10(c), the interface patch 900 is circular, but may alternatively be oval. The interface patch 900 of FIG. 10(c) may be any of the interface patches described above with reference to FIGS. 1 to 8. The interface patch 900 includes a central aperture 906. The interface patch 900 includes a pull tab 980 arranged on a peripheral edge. In this example, the interface patch 900 includes four pull tabs 980 even spaced about the peripheral edge of the interface patch 900. Each pull tab 980 acts as a pull tab region for removal of the interface patch 900 from the patient's skin. The interface patch 900 includes an adhesive region 968. The pull tabs 980 are non-adhesive. The adhesive region 968 extends across the surface of the interface patch 900, except for an area surrounding the aperture 906, but in other examples the adhesive region 968 may extend across the entire surface of the interface patch 900 except for the pull tabs 980. The adhesive of the adhesive region may be evenly applied or pattern-applied. The adhesive region 968 includes at least one region of reduced adhesion 972. The region of reduced adhesion 972 is located adjacent to a pull tab 980 and is substantially as described above with reference to FIG. 10(a). In particular, the region of reduced adhesion 972 has a first region 972a and lines 972b, 972c extending from the first region 972a as shown in FIG. 10(a). Preferably the lines 972b, 972c are divergent. Preferably, arms 972 extend from the lines 972b, 972c. In this illustrated example the interface patch 900 includes four regions of reduced adhesion 972—one positioned adjacent to each pull tab 980. As described above, the region(s) of reduced adhesion 972 may reduce skin irritation and damage when the interface patch 900 is peeled from the patient's skin.

It will be appreciated by persons skilled in the art that the above detailed examples have been described by way of example only and not in any limitative sense, and that various alterations and modifications are possible without departing from the scope of the invention as defined by the appended claims. Various modifications to the detailed examples described above are possible.

Through the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract or drawings), or to any novel one, or any novel combination, of the steps of any method or process so disclosed.

It will be appreciated by persons skilled in the art that the above embodiment(s) have been described by way of example only and not in any limitative sense, and that various alterations and modifications are possible without departing from the scope of the invention as defined by the appended claims. Various modifications to the detailed designs as described above are possible.

1. A subcutaneous medical device system, comprising an interface patch and a subcutaneous medical device having an adhesive surface and a needle for piercing a patient's skin, wherein the interface patch comprises:
a proximal side having an adhesive region for attaching the interface patch to the patient's skin;
a distal side to which the adhesive surface of the subcutaneous medical device is attachable; and
an aperture or pierceable region positioned to receive the needle of the subcutaneous medical device such that when the subcutaneous medical device is attached to the interface patch the needle can pierce the patient's skin through the aperture or pierceable region.

2. A subcutaneous medical device system according to claim 1, wherein the interface patch is larger than the adhesive surface of the subcutaneous medical device.

3. A subcutaneous medical device system according to claim 1 or claim 2, wherein the adhesive region on the proximal side of the interface patch has a larger surface area than the adhesive surface of the subcutaneous medical device.

4. A subcutaneous medical device system according to any one of the preceding claims, wherein the adhesion strength of the adhesive region on the proximal side is less than the adhesion strength of the adhesive surface of the subcutaneous medical device.

5. A subcutaneous medical device system according to any one of the preceding claims, wherein the adhesive region on the proximal side of the interface patch comprises a uniform adhesion surface.

6. A subcutaneous medical device system according to any one of the preceding claims, wherein the adhesive region on the proximal side of the interface patch is pattern-applied, for example comprising a line pattern or a grid pattern.

7. A subcutaneous medical device system according to any one of the preceding claims, wherein the adhesive region on the proximal side of the interface patch comprises at least one region of reduced adhesion.

8. A subcutaneous medical device system according to any one of the preceding claims, further comprising at least one removable release layer covering the adhesive region.

9. A subcutaneous medical device system according to claim 8, wherein the release layer is a single piece release layer.

10. A subcutaneous medical device system according to claim 9, wherein the single piece release layer comprises a first end at the edge of the proximal side and a second end at the aperture.

11. A subcutaneous medical device system according to claim 10, wherein the single piece release layer has a spiral form between the first end and the second end.

12. A subcutaneous medical device system according to claim 8, wherein the release layer comprises a plurality of sections.

13. A subcutaneous medical device system according to claim 12, wherein the release layer comprises a central section covering a central region of the proximal side of the interface patch, and a peripheral section covering an edge region of the proximal side of the interface patch.

14. A subcutaneous medical device system according to any one of claims 8 to 13, wherein the at least one release layer comprises a pull tab.

15. A subcutaneous medical device system according to any one of the preceding claims, further comprising at least one support layer attached to the distal side of the interface patch.

16. A subcutaneous medical device system according to claim 15, wherein the support layer is removable.

17. A subcutaneous medical device system according to any one of the preceding claims, wherein the needle extends in a direction perpendicular to the adhesive surface of the subcutaneous medical device.

18. A subcutaneous medical device system according to any one of the preceding claims, further comprising a cover attachable to the interface patch to cover the subcutaneous medical device.

19. A subcutaneous medical device system according to claim 18, wherein the cover is concave.

20. A subcutaneous medical device system, comprising an interface patch and a subcutaneous medical device having an adhesive surface and a needle for piercing a patient's skin, wherein the interface patch comprises:
a first portion having a proximal side with an adhesive for attaching the interface patch to the patient's skin, and a distal side to which the adhesive surface of the subcutaneous medical device can be attached such that when the subcutaneous medical device is attached to the interface patch the needle can pierce the patient's skin; and
a second portion, extending from the first portion and foldable to cover the subcutaneous medical device when the subcutaneous medical device is adhered to the first portion.

21. The subcutaneous medical device system of claim 20, wherein the first portion is joined to the second portion at a neck.

22. The subcutaneous medical device system of claim 21, wherein the neck is narrower than the first portion and the second portion.

23. The subcutaneous medical device system of any of claims 20 to 22, wherein the first portion has a generally triangular shape.

24. The subcutaneous medical device system of any of claims 20 to 23, wherein the second portion has a generally triangular shape.

25. The subcutaneous medical device system of claim 20, wherein the first portion has a generally triangular shape and the second portion has a generally triangular shape, and wherein the first portion and the second portion are joined at corners of the first and second portions such that the first and second portions have opposite orientations before the second portion is folded to cover the subcutaneous medical device.

26. The subcutaneous medical device system of any of claims 20 to 25, wherein the second portion is smaller than the first portion.

27. The subcutaneous medical device system of any of claims 20 to 26, wherein a distal side of the second portion comprises an adhesive for adhering to the distal side of the first portion when the second portion is folded to cover the subcutaneous medical device.

28. The subcutaneous medical device system of any of claims 20 to 27, wherein the second portion is flexible.

29. The subcutaneous medical device system of any of claims 20 to 28, further comprising a rigid cover arranged to cover the subcutaneous medical device when the second portion is folded to cover the subcutaneous medical device.

30. The subcutaneous medical device system of claim 29, wherein the rigid cover is attached to the second portion.

31. The subcutaneous medical device system of claim 29 or claim 30, wherein the rigid cover is concave.

32. The subcutaneous medical device system of any of claims 20 to 31, wherein the first portion comprises a pull tab region for removal of the first portion from the patient's skin, and a region of reduced adhesion disposed adjacent to the pull tab region.

33. The subcutaneous medical device system of claim 32, wherein the region of reduced adhesion comprises a first region disposed adjacent to the pull tab region, and at least one line extending from the first portion.

34. The subcutaneous medical device system of claim 33, wherein the region of reduced adhesion comprises first and second lines extending away from the first region in a divergent manner.

35. The subcutaneous medical device system of claim 32 or claim 33, wherein the region of reduced adhesion further comprises at least one arm extending from the at least one line.

36. The subcutaneous medical device system of any of claims 20 to 35, wherein the subcutaneous medical device comprises a device body and an adhesive patch for adhering the device body to the interface patch.

37. The subcutaneous medical device system of any of claims 20 to 36, further comprising an applicator for applying the subcutaneous medical device to the interface patch and patient.

38. A subcutaneous medical device system, comprising an interface patch and a subcutaneous medical device having an adhesive surface and a needle for piercing a patient's skin, wherein the interface patch comprises:
a proximal side with an adhesive region for attaching the interface patch to the patient's skin,
a distal side to which the adhesive surface of the subcutaneous medical device can be attached such that when the subcutaneous medical device is attached to the interface patch the needle can pierce the patient's skin; and
a pull tab region for removing the interface patch from the patient's skin;
wherein the adhesive region comprises a region of reduced adhesion comprising a first region disposed adjacent to the pull tab region and at least one line extending from the first region.

39. The subcutaneous medical device system of claim 38, wherein the region of reduced adhesion comprises a first line and a second line extending from the first region in a divergent manner.

40. The subcutaneous medical device system of claim 38 or claim 39, further comprising at least one arm extending from the or each line.

41. The subcutaneous medical device system of any of claims 38 to 40, wherein the region of reduced adhesion is adhesive-free.

42. The subcutaneous medical device system of any of claims 38 to 40, wherein the region of reduced adhesion comprises a lower adhesive thickness than the remainder of the adhesive region.

43. The subcutaneous medical device system of any of claims 38 to 42, wherein the adhesive region comprises a pattern-applied adhesive, for example a pattern-applied pressure sensitive adhesive.

44. The subcutaneous medical device system of any of claims 38 to 43, wherein the adhesive region extends to a peripheral edge of the interface patch.

45. The subcutaneous medical device system of any of claims 38 to 44, wherein the pull tab region is adhesive-free.

46. The subcutaneous medical device system of any of claims 38 to 45, wherein the pull tab region extends from an edge of the interface patch.

47. The subcutaneous medical device system of any of claims 38 to 46, comprising a plurality of pull tab regions and a plurality of regions of reduced adhesion, each region of reduced adhesion being disposed adjacent to one of the plurality of pull tab regions.

48. The subcutaneous medical device system of any of claims 38 to 47, wherein the interface patch has a polygonal shape, with sides and corners.

49. The subcutaneous medical device system of claim 48, wherein the interface patch has a triangular shape with three sides and three corners.

50. The subcutaneous medical device system of claim 48 or claim 49, wherein the pull tab region is formed at a corner of the interface patch.

51. The subcutaneous medical device system of claim 50, comprising a plurality of regions of reduced adhesion, each region of reduced adhesion being disposed adjacent to a corner of the interface patch, and wherein a plurality of pull tab regions are formed at the respective corners.

52. The subcutaneous medical device system of any of claims 38 to 51, further comprising an aperture for the needle of the subcutaneous medical device.

53. The subcutaneous medical device system of claim 52, comprising a non-adhesive region surrounding the aperture.

54. The subcutaneous medical device system of any of claims 38 to 53, wherein the interface patch comprises:
a first portion comprising the adhesive region, wherein the subcutaneous medical device can be adhered to the first patch portion, and
a second portion extending from the first portion and foldable to cover the subcutaneous medical device when the subcutaneous medical device is adhered to the first portion.

55. The subcutaneous medical device system of claim 54, wherein the first portion is joined to the second portion at a neck.

56. The subcutaneous medical device system of claim 54 or claim 55, wherein the first portion has a polygonal shape, for example a triangular shape.

57. The subcutaneous medical device system of any of claims 54 to 56, wherein the second portion is smaller than the first portion.

58. The subcutaneous medical device system of any of claims 54 to 57, wherein the second portion comprises an adhesive for adhering to the first portion when the second portion is folded over the first portion.

59. The subcutaneous medical device system of any of claims 38 to 58, wherein the subcutaneous medical device comprises a device body and an adhesive patch for adhering the device body to the interface patch.

60. The subcutaneous medical device system of any one of the preceding claims, wherein the subcutaneous medical device comprises a glucose sensor, for example a flash glucose sensor or a continuous glucose monitor.

61. The subcutaneous medical device system of any one of claims 1 to 59, wherein the subcutaneous medical device comprises an infusion set for delivery of a medicament to the patient.
